(19) United States
(12) Patent Application Publication
(10) Pub. No.: US 2026/0098270 A1
(43) Pub. Date: Apr. 9, 2026

(54) MULTIVALENT TRIDENT APTAMERS FOR MOLECULAR RECOGNITION, METHODS OF MAKING AND USES THEREOF

(71) Applicant: MCMASTER UNIVERSITY, Hamilton (CA)

(72) Inventors: Yingfu LI, Dundas (CA); John BRENNAN, Dundas (CA); Jiuxing LI, Hamilton (CA); Zijie ZHANG, Hamilton (CA); Jimmy GU, Mississauga (CA); Ryan AMINI, Hamilton (CA); Alexandria MANSFIELD, Hamilton (CA); Jianrun XIA, Ancaster (CA); Dawn WHITE, Barrie (CA); Hannah D. STACEY, St. Marys (CA); Jann C. ANG, Mississauga (CA); Gurpreet PANESAR, Hamilton (CA); Alfredo CAPRETTA, Dundas (CA); Carlos FILIPE, Ancaster (CA); Karen MOSSMAN, Ancaster (CA); Bruno J. SALENA, Ancaster (CA); Cynthia BALION, Hamilton (CA); Leyla SOLEYMANI, Oakville (CA); Matthew S. MILLER, Hamilton (CA); Deborah YAMAMURA, Burlington (CA); Sam AFKHAMI, Oakville (CA); Michael Rein D'AGOSTINO, Toronto (CA)

(73) Assignee: MCMASTER UNIVERSITY, Hamilton, ON (CA)

(21) Appl. No.: 19/113,866

(22) PCT Filed: Sep. 22, 2023
(86) PCT No.: PCT/CA2023/051253

§ 371 Date: Mar. 20, 2025

Related U.S. Application Data
(60) Provisional application No. 63/409,455, filed on Sep. 23, 2022.
(60) Provisional application No. 63/527,795, filed on Jul. 19, 2023.
(60) Provisional application No. 63/515,519, filed on Jul. 25, 2023.

(51) Int. Cl. C12N 15/115; A61K 9/00; A61K 31/7088; A61P 31/16; C12N 15/113; C12Q 1/68; C12Q 1/70

(52) U.S. Cl. CPC C12N 15/115; A61K 9/0043; A61K 31/7088; A61P 31/16; C12N 15/1131; C12Q 1/68; C12Q 1/701; C12N 2310/16; C12N 2310/51

(57) ABSTRACT

Multivalent trident aptamers comprising the general formula [A-S_{A}]_{2 or 3}-L-[S_{B}-B] in which a central branched linker molecule (L) possesses 2 or 3 variable arms ([A-S_{A}]_{2 or 3}), and a root ([S_{B}-B]), connected by a central carbon atom, to provide enhanced affinity and/or avidity with a target are described, as well as methods of making and using the multivalent trident aptamers are provided.

### FIELD OF THE INVENTION

The present invention relates to the field of aptamers and, in particular, to multivalent aptamer constructs having a trident formation, and methods of making and using thereof.

### BACKGROUND OF THE INVENTION

Aptamers are short single-stranded RNA or DNA molecules capable of specific binding to various molecular targets, such as small molecules, proteins, nucleic acids, and even cells and tissues, due to the formation of characteristic spatial structures. Aptamers have been described as the chemical equivalent of antibodies, however, aptamers have the advantage of being highly specific, relatively small in size, non-immunogenic, and easily synthesized in quantity with high purity. For these reasons, it is believed that aptamers will find broad application in biosensing, bioanalysis, biotechnology, and biomedicine.

A number of approaches have been taken for generating aptamers designed to acquire a range of desired features, for example improved stability, target binding affinity, and conjugation to reporter groups, cell-toxic molecules, nanoparticles, etc.

Among the large variety of aptamer-based constructs, multimeric aptamers have been described that comprise two or more identical or different aptamer motifs, with or without additional structural elements or functional groups. It has been described, for example, that simple concatenation of the same aptamer motif can significantly improve the avidity of a construct due to multiple target binding sites. Other combinations of different aptamer motifs are also believed to offer opportunities to build multifunctional molecules.

Nonetheless, it remains a current objective in aptamer-based research and development to obtain aptamer constructs that are effective molecular recognition elements (MREs) that can be used for different bioanalytical and therapeutic applications.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide multivalent trident aptamers for molecular recognition, and methods of making and uses thereof. In accordance with one aspect of the invention, there is provided a multivalent trident aptamer comprising the general formula: [A-S_{A}]_{2 or 3}-L-[S_{B}-B], wherein: A is an aptamer specific for one or more epitopes of a target molecule or protein, ranging from 5 to 150 nucleotides in length; L is a linker comprising up to three branches and a root, wherein each branch comprises a terminal end that links to one of the [A-S_{A}]s to form a trident configuration; S_{A} is a spacer molecule that separates each A from the L-branch at a distance of up to 20.5 nm; B is a functional molecule selected from an aptamer, a reporter molecule, or a crosslinker that is linked to the terminal end of the root of L; and S_{B} is a spacer molecule that separates B from the L-root at a distance of up to 20.5 nm.

In accordance with another aspect of the invention, there is provided a multivalent trident aptamer comprising the general formula: [A-S_{A}]₃-L-[S_{B}-B], wherein: A is an aptamer specific for one or more epitopes of a target molecule or protein, ranging from 5 to 150 nucleotides in length; L is a linker comprising up to three branches and a root, wherein each branch comprises a terminal end that links to one of the [A-S_{A}]s to form a trident configuration; S_{A} is a spacer molecule that separates each A from the L-branch at a distance of up to 20.5 nm; B is a functional molecule selected from an aptamer, a reporter molecule, or a crosslinker that is linked to the terminal end of the root of L; and S_{B} is a spacer molecule that separates B from the L-root at a distance of up to 20.5 nm.

In some embodiments, the trident aptamer is a symmetrical trident construct. In certain embodiments, the trident aptamer is a symmetric trebler biomolecule. In other embodiments, the trident aptamer is a symmetric doubler biomolecule. In further embodiments, the trident aptamer is a trebler phosphoramidite.

In some embodiments, S_{A} comprises a single stranded nucleic acid sequence of 0 to 30 nucleotides. In certain embodiments, S_{A} is a single stranded thymine-rich sequence of 12 nucleotides. In other embodiments, S_{A} comprises up to 15 linear alkane chains each comprising up to 12 carbon atoms. In further embodiments, S_{A} comprises a linear polyethylene glycol chain of up to 55 ethylene glycol units.

In some embodiments, B is an aptamer identical to A. In other embodiments, B is a reporter molecule selected from an antigen, an enzyme, and a fluorescent molecule. In further embodiments, B is a crosslinker selected from thiol, amide, biotin, digoxigenein, azide, alkyne, carboxyl, and a Click-Chemistry-based crosslinker.

In some embodiments, S_{B} comprises a single stranded nucleic acid sequence of 0 to 30 nucleotides. In other embodiments, S_{B} is a single stranded thymine sequence of 5 nucleotides. In further embodiments, S_{B} comprises up to 15 linear alkane chains each comprising up to 12 carbon atoms. In other embodiments, S_{B} comprises a linear polyethylene glycol chain of up to 55 ethylene glycol units.

In some embodiments, the trident aptamer is a homo-trimeric aptamer comprising identical As. In certain embodiments, the As are in the same 5′ to 3′ orientation relative to L. In other embodiments, the As are in the same 3′ to 5′ orientation relative to L.

In some embodiments, the trident aptamer is a hetero-trimeric aptamer comprising As that are each specific to a different epitope of a target molecule or protein.

In some embodiments, A is in a range of 20 to 90 nucleotides in length. In certain embodiments, the target molecule is SARS-COV-2 spike protein and A is MSA52. In other embodiments, the target molecule is SARS-COV-2 S protein and A is MSA52T8. In other embodiments, the target molecule is Influenza HA and A is RHA06. In further embodiments, the target molecule is VEGF165 and A is H1A. In other embodiments, the target molecule is Troponin I and A is TnAp1.

In accordance with another aspect of the invention, there is provided a pharmaceutical composition comprising a multivalent trident aptamer comprising the general formula: [A-S_{A}]_{2 or 3}-L-[S_{B}-B], wherein: A is an aptamer specific for one or more epitopes of a target molecule or protein, ranging from 5 to 150 nucleotides in length; L is a linker comprising up to three branches and a root, wherein each branch comprises a terminal end that links to one of the [A-S_{A}]s to form a trident configuration; S_{A} is a spacer molecule that separates each A from the L-branch at a distance of up to 20.5 nm; B is a functional molecule selected from an aptamer, a reporter molecule, or a crosslinker that is linked to the terminal end of the root of L; and S_{B} is a spacer molecule that separates B from the L-root at a distance of up to 20.5 nm; and one or more pharmaceutically acceptable excipients.

In accordance with another aspect of the invention, there is provided a use of a multivalent trident aptamer comprising the general formula: [A-S_{A}]_{2 or 3}-L-[S_{B}-B], wherein: A is an aptamer specific for one or more epitopes of a target molecule or protein, ranging from 5 to 150 nucleotides in length; L is a linker comprising up to three branches and a root, wherein each branch comprises a terminal end that links to one of the [A-S_{A}]s to form a trident configuration; S_{A} is a spacer molecule that separates each A from the L-branch at a distance of up to 20.5 nm; B is a functional molecule selected from an aptamer, a reporter molecule, or a crosslinker that is linked to the terminal end of the root of L; and S_{B} is a spacer molecule that separates B from the L-root at a distance of up to 20.5 nm; in the preparation of a medicament for the targeted delivery of a drug in a subject.

In accordance with another aspect of the invention, there is provided a biosensor comprising a multivalent trident aptamer comprising the general formula: [A-S_{A}]_{2 or 3}-L-[S_{B}-B], wherein: A is an aptamer specific for one or more epitopes of a target molecule or protein, ranging from 5 to 150 nucleotides in length; L is a linker comprising up to three branches and a root, wherein each branch comprises a terminal end that links to one of the [A-S_{A}]s to form a trident configuration; S_{A} is a spacer molecule that separates each A from the L-branch at a distance of up to 20.5 nm; B is a functional molecule selected from an aptamer, a reporter molecule, or a crosslinker that is linked to the terminal end of the root of L; and S_{B} is a spacer molecule that separates B from the L-root at a distance of up to 20.5 nm; immobilized on and/or in a material.

In accordance with another aspect of the invention, there is provided a method for detecting the presence of a target molecule in a sample, the method comprising: a. contacting the sample with a multivalent trident aptamer comprising the general formula: [A-S_{A}]_{2 or 3}-L-[S_{B}-B], wherein: A is an aptamer specific for one or more epitopes of a target molecule or protein, ranging from 5 to 150 nucleotides in length; L is a linker comprising up to three branches and a root, wherein each branch comprises a terminal end that links to one of the [A-S_{A}]s to form a trident configuration; S_{A} is a spacer molecule that separates each A from the L-branch at a distance of up to 20.5 nm; B is a functional molecule selected from an aptamer, a reporter molecule, or a crosslinker that is linked to the terminal end of the root of L; and S_{B} is a spacer molecule that separates B from the L-root at a distance of up to 20.5 nm, wherein the multivalent trident aptamer binds the target molecule; and b. detecting the binding of the multivalent trident aptamer with the target molecule.

### BRIEF DESCRIPTION OF THE DRAWINGS

These and other features of the invention will become more apparent in the following detailed description in which reference is made to the appended drawings.

FIG. 1 shows an assessment of the binding affinity of monomeric and trident RHA06 for influenza HA proteins using a dot blot assay. (A) Side-by-side comparison of representative dot blot results and (B) binding curves used to derive the K_{d} values and affinity enhancement folds. Trident RHA06 with control protein (BSA) and mutant trident aptamer with H3N2 were also included as controls. BA: bound aptamer; UA: unbound aptamer.

FIG. 2 shows an assessment of the binding affinity of H1A and trident H1A aptamers for VEGF165 using a dot blot assay. (A) Dot blot results and (B) Binding curves used to derive the K_{d} values. Affinity enhanced ˜78-fold.

FIG. 3 shows an assessment of the binding affinity of TnAp1 and trident TnAp1 aptamers for troponin I using a dot blot assay. Affinity enhanced ˜30-fold.

FIG. 4 shows an assessment of the binding affinity of MSA52T8 and trident TMSA52T8 for S1 of SARS-COV-2 using a dot blot assay. (A) Dot blot results and (B) binding curves used to derive the K_{d} values.

FIG. 5 shows an assessment of the cooperative binding effect of trimeric H3N2-HA protein by the three branches of trident RHA06 aptamer by adding antisense sequence (AS) of RHA06. K_{d} increased indicating reduced affinity. The three branches of trident RHA06 were determined bound with the trimeric HA protein.

FIG. 6 shows an assessment of the cooperative binding effect of VEGF₁₆₅ by three branches of trident H1A aptamer using antisense sequence (AS) of H1A. K_{d} increased indicating reduced affinity. The three branches of trident H1A were determined to be bound with VEGF protein, though VEGF was determined to be a dimeric protein.

FIG. 7 shows an assessment of the cooperative binding effect of S1 by three aptamer branches of TMSA52T8 using antisense sequence (AS) of MSA52T8. K_{d} increased indicating the three branches of TMSA52T8 bound with S1 protein.

FIG. 8 shows a BLI kinetic analysis of RHA06 and trident RHA06 binding with the trimeric influenza H3N2-HA protein. K_{d} values consistent with the dot blot results. kₒₙ an k_{off} indicated the association and dissociation rates.

FIG. 9 shows an assessment of the binding affinity of heteromeric aptamer, homomeric aptamer (TMSA52T8), and monomeric aptamer (MSA52T8) for S-protein of SARS-COV-2 using a dot blot assay. (A) Dot blot results and (B) binding curves used to derive the K_{d} values.

FIG. 10 shows a design of a trimeric aptamer for the trimeric spike protein of SARS-COV-2 in exemplary embodiments of the disclosure: (A) Top view of the SARS-CoV-2 trimeric spike protein showing the complementarity with the trimeric aptamer to achieve molecular recognition. The trimeric structure of the aptamer ligand complements the molecular scaffold of the spike protein and optimizes binding affinity; (B) Secondary structure of SARS-COV-2 spike protein binding aptamer MSA52. The ability of MSA52 to universally identify ongoing and predicted SARS-COV-2 variants of concern provides an ideal candidate for COVID-19 detection; (C) Construction of the trimeric aptamer with MSA52, trebler, and 15-thymine linker. The trebler biomolecule enables the assembly of a symmetric, multimeric recognition element.

FIG. 11 shows a size analysis of TMSA52, DMSA52 and MSA52 using dPAGE in exemplary embodiments of the disclosure. The DNA sequences are listed in Table 1. The mobility of linear monomeric aptamer (MSA52) and linear dimeric aptamer (DMSA52) are consistent with DNA ladder. The mobility of branched trimeric aptamer (TMSA52) is significantly slower than the corresponding DNA ladder due to the branched structure. The result showed that branched trimeric aptamer (TMSA52) was synthesized successfully.

FIG. 12 shows an assessment of the binding affinity of trimeric aptamer in exemplary embodiments of the disclosure: (A) the wild-type (WT), B.1.1.7, B.1.351, and P. 1 spike protein variants; (B) the B.1.429, B.1.617.1, B.1.617.2, and B.1.1.529 spike protein variants; (C) SARS-COV-1 spike protein, and RBD of seasonal coronavirus 229E and OC43 by dot-blot assays; (D) The corresponding plots of bound fraction against protein concentrations in panel (C) show the binding affinity of trimeric aptamer for SARS-COV-1 spike protein, and RBD of seasonal coronavirus 229E and OC43. BA: bound aptamer; UA: unbound aptamer.

FIG. 13 shows an assessment of binding affinity of TMSA52 in exemplary embodiments of the disclosure: (A) SARS-COV-2 spike protein variants; (B) pseudotyped lentiviruses displaying the spike protein variants. K_{d} values displayed consistently high affinity (pM to fM range) for both WT and variant strains.

FIG. 14 shows an assessment of the binding affinity of trimeric aptamer in exemplary embodiments of the disclosure: (A) the pseudotyped lentiviruses expressing the wild-type (WT), B.1.1.7, B.1.351, and P.1 spike protein variants; (B) the pseudotyped lentiviruses expressing the B.1.429, B.1.617.1, B.1.617.2, and B.1.1.529 spike protein variants; (C) control lentiviruses by dot-blot assays; (D) The corresponding plot of bound fraction against virus concentrations in panel (C) shows the binding affinity of trimeric aptamer for control lentiviruses. BA: bound aptamer; UA: unbound aptamer.

FIG. 15 shows validation of the cooperative effect amongst the aptamer arms of TMSA52 for the binding of spike protein in exemplary embodiments of the disclosure: (A) Blocking of trimeric aptamer (TMSA52) arms using various AS:TMSA52 ratios. AS is a 40-nt single-stranded DNA sequence that is complementary to aptamer MSA52; (B) Electrophoretic mobility shift assay; (C) corresponding plot displaying the hybridization efficiency between ³²P-AS and TMSA52 at different AS:TMSA52 ratios.

FIG. 16 shows the cooperativity of binding by three aptamer arms in exemplary embodiments of the disclosure: (A) Dot-blot assays; (B) corresponding binding affinity curves of TMSA52 for B.1.1.529 spike protein at different AS:TMSA52 ratios. BA: bound aptamer; UA: unbound aptamer.

FIG. 17 shows an assessment of binding affinity in exemplary embodiments of the disclosure: (A) monomeric aptamer (MSA52); (B) dimeric aptamer (DMSA52) for B.1.1.529 S by dot-blot assays; (C) Plots of bound fraction against B.1.1.529 S concentrations in panels (A) and (B) showing the binding affinity of monomeric aptamer (MSA52) and dimeric aptamer (DMSA52) for B.1.1.529 S. BA: bound aptamer; UA: unbound aptamer.

FIG. 18 shows a design of an enzyme-linked aptamer binding assay for colorimetric detection of SARS-COV-2 using TMSA52 in exemplary embodiments of the disclosure: (A) Illustration of colorimetric sandwich assay in which SARS-COV-2 pseudoviruses are captured with biotinylated aptamers, using Pd—Ir nanocubes as peroxidase-mimicking nanozymes; S_{A}: streptavidin: (B) Photograph of colorimetric test; (C) corresponding concentration-response plots using A₄₅₀ for pseudotyped lentivirus expressing the SARS-COV-2 B.1.1.529 spike protein in buffer, using the biotinylated trimeric aptamer (TMSA52-B) at different AS:TMSA52-B ratios; AS is a 40-nt single-stranded DNA sequence that is complementary to aptamer MSA52; (D) Response based on A₄₅₀ (inset: photograph) demonstrating the specificity of the trimeric aptamer-based method for the detection of B.1.1.529 pseudovirus. SARS-COV-1 spike protein, spike RBD of seasonal coronaviruses 229E and OC43, human IgG, amylase, BSA, and lentivirus are used as controls. The concentrations of control proteins were 10 nM; the concentrations of pseudovirus and lentivirus were 10⁵ cp/mL (corresponding to 120 aM). LOD: limit of detection, 3 times the standard deviation of blank samples.

FIG. 19 shows TEM characterization of Pd—Ir nanocubes in exemplary embodiments of the disclosure.

FIG. 20 shows a photograph and corresponding plots of absorbance in exemplary embodiments of the disclosure: (A) The photograph: (B) corresponding plots of absorbance at 450 nm showing the detection of the pseudotyped lentiviruses expressing the B.1.1.529 spike protein variant by biotinylated monomeric aptamer (MSA52-B), dimeric aptamer (DMSA52-B), and trimeric aptamer (TMSA52-B). LOD: limit of detection, 3 times the standard deviation of blank samples.

FIG. 21 shows the detection of pseudoviruses spiked human saliva in exemplary embodiments of the disclosure: (A) Photograph of colorimetric test; (B and C) corresponding plots depicting the detection of pseudoviruses expressing different spike protein variants spiked in pooled human saliva using the Enzyme-Linked Aptamer Binding Assay (ELABA) assay with TMSA52. LOD: limit of detection, 3 times the standard deviation of blank samples.

FIG. 22 shows clinical evaluation of the trimeric aptamer assay in exemplary embodiments of the disclosure: (A) Schematic illustration; (B) signal response of the TMSA52-based ELABA for the detection SARS-COV-2 in clinical saliva samples, including 50 NPS positive samples (wild-type SARS-COV-2, Alpha (B.1.1.7), Gamma (P.1), Delta (B.1.617.2), Omicron (B.1.1.529: PS9-PS21, BA.2: PS22-PS23, BA.4: PS24-PS26, BA.5: PS27-PS36, BA.2.12.1: PS37-PS44) and undetermined variants) and 60 NPS negative samples. PS: positive saliva sample; NS: negative saliva sample; PC: positive control, 4×10⁴ cp/mL B.1.1.529 pseudovirus spiked in commercial pooled human saliva; NC: negative control, commercial pooled human saliva. The dotted line marks the cut-off point for the TMSA52-based assay. The error bars stand for the standard deviation of three (n=3) replicated samples. ND: not detected. NPS: nasopharyngeal swab.

FIG. 23 shows a Receiver Operating Characteristic (ROC) curve to determine the clinical cut-off absorbance value in exemplary embodiments of the disclosure: (A) Box and whisker plot depicting the distribution of the NPS positive and negative patient saliva samples presented in FIG. 12B; (B) Receiver-Operator Characteristics Curve for the TMSA52-based ELABA. The overall accuracy (AUC: Area Under the Curve) was 0.929 (95% confidence interval: 0.875-0.983) with an optimum sensitivity of 84.0% (true positive cases detected) and a corresponding specificity of 98.3% (true negative cases detected) at a threshold absorbance (450 nm) of 0.027.

FIG. 24 shows BTNX COVID-19 antigen rapid test for the detection SARS-CoV-2 in clinical saliva samples in exemplary embodiments of the disclosure. Including 50 NPS positive samples and 60 NPS negative samples (grey). PS: Positive saliva sample; NS: Negative saliva sample. C: control line; T: test line; NPS: nasopharyngeal swab; (+) positive; (−) negative.

FIG. 25 shows percent neutralization of SARS-COV-2 Omicron BA.1 (A) or MA10 (B) by aptamer constructs or S309 antibody in vitro on Vero E6 cells at 3 days post-infection to assess the aptamers ability to neutralize SARS-COV-2 in vitro. Briefly, serial dilutions of TMSA52 (1 μM), Ir-TMSA52 (1 μM), Ir (1 μM), and S309 (1 μg/mL) were incubated with the virus, and the mixture was plated on Vero E6 cells for 3 days prior to measuring the cell viability using Cell Titre Glo 2.0. Open circles represent TMSA52 (universal SARS-COV-2 aptamer), closed circles represent Ir-TMSA52 (universal SARS-CoV-2 aptamer on an iridium nanoplate), open triangles represent Ir (Iridium nanoplate), closed triangles represent S309 (SARS-COV-2 monoclonal neutralizing antibody).

FIG. 26 shows the in vivo assessment of aptamers in protection against non-lethal challenge with SARS-COV-2. (A) illustrates the experimental schema. Animals were treated intranasally (i.n.) with either nuclease free water, TMSA52 (universal SARS-COV-2 aptamer), Ir-TMSA52 (universal SARS-COV-2 aptamer on an iridium nanoplate), Ir (iridium nanoplate), S309 (SARS-COV-2 monoclonal neutralizing antibody), or mTMSA52 (a mutated TMSA52 with partially abrogated binding to SARS-COV-2 spike) 2 hours prior to challenge with a sub-lethal dose (10⁴ PFU) of SARS-COV-2 MA10. Animals were subsequently monitored for weight loss and mortality for 7 days. A cohort of animals (n=5) was sacrificed 4 days post-infection for determination of lung viral burden. (B) shows the weight loss following treatment, in comparison to nuclease-free water treated controls. (C) shows the viral burden (Log₁₀ TCID₅₀) in the lung at 4 days post-SARS-COV-2 infection. Briefly, lungs were homogenized, and plated on Vero E6 cells for 5 days before enumeration by visually assessing cytopathic effect. Closed squares represent control animals, open circles represent TMSA52, closed circles represent Ir-TMSA52, open triangles represent Ir, closed triangles represent S309, open squares represent mutant aptamer. Data is represented mean±SEM. Statistical analysis for panel E was a one-way ANOVA with Tukey multiple comparisons test. Data is representative of 1 independent experiment, ns=not significant.

FIG. 27 shows the in vivo assessment of aptamers in protection against lethal challenge with SARS-COV-2. (A) illustrates the experimental schema. Animals were treated intranasally (i.n.) with either nuclease free water, TMSA52 (universal SARS-COV-2 aptamer), Ir-TMSA52 (universal SARS-COV-2 aptamer on an iridium nanoplate), Ir (iridium nanoplate alone), S309 (SARS-COV-2 monoclonal neutralizing antibody), or scrambled aptamer (a scrambled TMSA52 with equimolar A:T and C:G ratios with abrogated binding to SARS-COV-2 spike) 2 hours prior to challenge with a lethal dose (10⁵ PFU) of SARS-CoV-2 MA10. Animals were subsequently monitored for weight loss and mortality for 7 days. A cohort of animals (n=5) was sacrificed 4 days post-infection for determination of lung viral burden and histopathology. (B) shows the weight loss following treatment, in comparison to nuclease-free water treated controls. (C) shows the Kaplan-Meier survival curve depicting percent-survival over the course of infection. Closed squares represent control animals, open circles represent TMSA52, closed circles represent Ir-TMSA52, open triangles represent Ir, closed triangle represent S309, open squares represent scrambled aptamer. (D) shows the clinical scoring. The number indicates the total number of clinical manifestations (fur ruffling, rapid respiration, lethargy, and/or hunched posture). (E) shows the gross histopathological representative images of lungs collected 4 days post-SARS-COV-2 infection. Arrows indicate areas of diffuse hemorrhage. (F) shows the viral burden (Log₁₀ TCID₅₀) in the lung at 4 days post-SARS-COV-2 infection. Briefly, lungs were homogenized, and plated on Vero E6 cells for 5 days before enumeration by visually assessing cytopathic effect. Closed squares represent control animals, open circles represent TMSA52, closed circles represent Ir-TMSA52, open triangles represent Ir, closed triangle represent S309, open squares represent scrambled aptamer. Data is represented mean±SEM. Statistical analysis for panel F was a one-way ANOVA with Tukey multiple comparisons test. Data is representative of 1 independent experiment, ns=not significant.

### DETAILED DESCRIPTION OF THE INVENTION

The present invention provides multivalent aptamer constructs having a trident formation, and their use as molecular recognition elements (MREs) that can be used for different bioanalytical and therapeutic applications. According to embodiments, the multivalent trident aptamer is designed for the arrangement of individual DNA or RNA aptamers in a trident structure for the purposes of increasing target binding affinity and avidity via the arrangement of homomeric or heteromeric aptamers with controlled orientation (5′-3′ or 3′-5′) and spacing from a central branched linker molecule to bind homomeric or heteromeric epitopes on a target.

In certain embodiments, the trident aptamer construct possesses the general structure [A-S_{A}]_{2 or 3}-L-[S_{B}-B]. Where a central branched (trebler or doubler) linker molecule (L) possesses 3 or 4 variable arms ([A-S_{A}]_{2 or 3}, [S_{B}-B]) connected by a central carbon atom. Three identical aptamer (A) and spacer (S_{A}) unit ([A-S_{A}]) branches can be attached to three of the four branches of ‘L’. According to such embodiments, the present invention provides high-binding affinity multi-armed aptamers with a form factor (trident) that allows for unimpeded folding of individual aptamers, optimizable direction, and alignment of arms that produces a high concentration, reducing dissociation off-rate. According to embodiments, the high concentration of spatially aligned aptamer units ensures target binding by at least one of the individual aptamer units (A) at each end of the trident arms. In certain embodiments, detachment of an individual aptamer unit can be replaced by one or more of the cooperating aptamer units (A) of the trident construct. In this way, the target is bound by at least one aptamer unit (A) of the trident construct at any given time. In some embodiments, the target is bound by two aptamer units (A) of the trident construct. In further embodiments, the target is bound by three aptamer units (A) of the trident construct.

Some embodiments of the invention provide for enhanced affinity and avidity. According to such embodiments, the trident form of the multiple individual homomeric or heteromeric aptamers enhance the avidity of the binding interaction by simultaneously binding multiple epitopes of a target. In certain embodiments, the trident construct design uses homomeric aptamers on the trident arms to bind targets with a single epitope. In other embodiments, multiple identical epitopes or heteromeric aptamers are presented on the arms to bind targets with different epitopes.

Other embodiments of the invention provide for enhanced folding consistency. As compared to concatemers (consecutive aptamer units on a single strand), for example, trident aptamers of the present invention may be more likely to fold consistently due to each aptamer element being immobilized at a single terminal. In contrast, internal aptamers of concatemers may be more restricted in folding due to the additional bulk of aptamer units up and downstream.

Further embodiments of the present invention provide for orientation control. Given that aptamers and their targets are known to bind in a preferred relative orientation, trident aptamer constructs of the present invention allow for the attachment of homomeric or heteromeric aptamer units in either the 5′-3′ or 3′-5′ orientation relative to the central linker molecule. This allows for the aptamers to be arranged in an optimal orientation for epitope binding. According to other embodiments, the present invention further allows for reversible aptamer orientation which is contemplated to allow for the relocation of the linker molecule relative to the target to facilitate secondary target binding.

In other embodiments, the present invention provides for optimizable aptamer spacing. In such embodiments, the spacing of the aptamer from the target epitope can be optimized using either linear carbon linker molecules or unstructured nucleic acids. Linker lengths can be designed to allow sufficient reach such that each aptamer can reach its respective epitope on the target, without being excessively long such that aptamer folding is inhibited or steric inhibition is observed. As well, for applications where binding clusters of epitopes on a target is desired, linker lengths can be restricted to limit binding to a local area (i.e., a single target molecule on a cell surface with multiple targets).

According to further embodiments, the present invention provides a multipurpose attachment site on the 3′ branch/arm of the trebler linker. In such embodiments, the 3′ arm may be functionalized with a variety of other molecules to enable i) surface immobilization or crosslinking via biotin, amine, thiol or digoxigenin molecules, ii) attachment of reporter elements such as fluorophores, enzymes (HRP), nucleic acid amplification primers (RCA, PCR, LAMP, etc.) or iii) drug delivery such as small molecule drugs, antimicrobial agents, and gene therapeutics.

### Definitions

Unless defined otherwise, all technical and scientific terms used herein have the same meaning as commonly understood by one of ordinary skill in the art to which this invention belongs.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used in this disclosure, the singular forms “a”, “an” and “the” include plural references unless the content clearly dictates otherwise.

The term “and/or” as used herein means that the listed items are present, or used, individually or in combination. In effect, this term means that “at least one of” or “one or more” of the listed items is used or present.

The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

The term “target”, “analyte” or “target analyte” as used herein refer to any agent, including, but not limited to, a small inorganic molecule, small organic molecule, metal ion, biomolecule, toxin, biopolymer (such as a nucleic acid, carbohydrate, lipid, peptide, protein), cell, tissue, microorganism and virus, for which one would like to sense or detect. The analyte can be either isolated from a natural source or synthetic. The analyte can be a single compound or a class of compounds, such as a class of compounds that share structural or functional features. The term analyte also includes combinations (e.g. mixtures) of compounds or agents such as, but not limited, to combinatorial libraries and samples from an organism or a natural environment.

The term “subject” as used herein includes all members of the animal kingdom including mammals such as a mouse, a rat, a dog, and a human.

The term “nucleic acid” as used herein refers to a biopolymer comprising monomers of nucleotides, such as deoxyribonucleic acid (DNA), ribonucleic acid (RNA) and other polynucleotides of modified nucleotides and/or nucleotide derivatives and can be either double stranded (ds) or single stranded (ss). In some embodiments, modified nucleotides contain one or more modified bases (e.g., unusual bases such as inosine, and functional modifications to the bases such as amino), modified backbones (e.g., peptide nucleic acid, PNA) and/or other chemically, enzymatically, or metabolically modified forms.

The term “aptamer” as used herein refers to a short, chemically synthesized nucleic acid molecule or oligonucleotide sequence which can be generated by in vitro selection to fold into specific three-dimensional structures that bind to a specific analyte with dissociation constants, for example, in the pico- to nano-molar range. Aptamers can be single-stranded DNA, RNA, modified nucleotides and/or nucleotide derivatives. Aptamers can also be naturally occurring RNA aptamers termed “riboswitches”. Functional aptamer sequences can also be rationally designed, truncated, conjugated, or otherwise modified from original parent (or full length) sequences. A functional fragment of an aptamer is the portion of an aptamer that retains aptameric function, for example, function in binding to molecules such as protein, lipid, carbohydrate, and nucleic acid. A functional variant of an aptamer refers to an aptamer that has been modified, with nucleotide derivates or otherwise elongated or truncated, and still retains aptameric function.

The term “hybridizes”, “hybridized” or “hybridization” as used herein refers to the sequence specific non-covalent binding interaction with a complementary, or partially complementary, nucleic acid sequence. When, for example, the 5′-end region of an aptamer hybridizes to the 3′-end region, it can form a duplex DNA element.

The term “affinity” as used herein refers to an interaction between one aptamer domain with its binding site that may be assessed by corresponding dissociation constant, K_{d}. The term “avidity” as used herein refers to the overall strength of multiple binding interactions and can be described by the K_{d} of the completely associated aptamer-target complex.

The term “arm(s)” and “branch(es)” are used interchangeably herein when referring to the arms or branches of a linker (L).

The terms “attenuate”, “inhibit”, “prevent”, “treat”, and grammatical variations thereof, as used herein, refer to a measurable decrease in a given parameter or event.

The term “treatment method”, or “method for the treatment of a pathology or disorder”, means therapy aimed at restoring the health condition of a subject, maintaining the existing condition and/or preventing the worsening of said health condition.

The term “trident” as used herein refers to the general visual configuration of the multivalent aptamers described herein in which the individual aptamer elements [A-S_{A}]_{2 or 3} that extend from each variable branch or arm of the branched linker molecule (L) are oriented in the same direction and in opposite direction from the root [S_{B}-B] element to form a trident or fork-like formation.

### Multivalent Trident Aptamers

Multivalent trident aptamers are provided that comprise the arrangement of individual DNA or RNA aptamers in a trident structure. The trident aptamer construct will possess the general formula [A-S_{A}]_{2 or 3}-L-[S_{B}-B] in which a central branched linker molecule (L) possesses 2 or 3 variable arms ([A-S_{A}]_{2 or 3}), and a root ([S_{B}-B]), connected by a central carbon atom.

According to certain embodiments, the trident aptamer construct possesses 2 variable arms and a root ([A-S_{A}]₂-L-[S_{B}-B]). In such embodiments, the central branched linker (L) can be a doubler to form a pitch-forked construct. In some embodiments the trident aptamer is a symmetric doubler construct comprising two identical aptamer and spacer units ([A-S_{A}]₂) attached to the two variable arms of the linker ‘L’. In other embodiments, the trident aptamer is an asymmetric doubler construct comprising two or three non-identical aptamer-spacer units attached to the two variable arms of the linker ‘L’.

According to certain embodiments, the trident aptamer construct possesses 3 variable arms and a root ([A-S_{A}]₃-L-[S_{B}-B]). In such embodiments, the central branched linker (L) can be a trebler to form the trident construct. In some embodiments the trident aptamer is a symmetric trebler construct comprising three identical aptamer and spacer units ([A-S_{A}]₃) attached to the three variable arms of the linker ‘L’. In other embodiments, the trident aptamer is an asymmetric trebler construct comprising two or three non-identical aptamer-spacer units attached to the three variable arms of the linker ‘L’.

In preferred embodiments, the trident aptamer construct possesses 3 variable arms to form a trimeric construct. According to such embodiments, three aptamer (A) and spacer (S_{A}) units ([A-S_{A}]₃) can be attached to the three variable branches of the linker ‘L’. In some embodiments the trident aptamer is a symmetric trebler construct comprising three identical aptamer and spacer units ([A-S_{A}]₃) attached to the three branches of the linker ‘L’. According to other embodiments, the trident aptamer is an asymmetric trebler construct comprising three non-identical aptamer-spacer units attached to the three branches of the linker ‘L’. In some embodiments the three branches of the linker ‘L’ are each attached to a different aptamer-spacer unit. In other embodiments two of the three branches of the linker ‘L’ are each attached to an identical aptamer-spacer unit. In a further embodiment, all three of the branches of the linker ‘L’ are each attached to an identical aptamer-spacer unit.

According to further embodiments, the doubler or trebler linker ‘L’ comprises a multipurpose attachment site on the 3′ arm of the linker ([S_{B}-B]). In such embodiments, the 3′ arm may be functionalized with a variety of other molecules to enable i) surface immobilization or crosslinking via biotin, amine, thiol or digoxigenin molecules, for example ii) attachment of reporter elements such as fluorophores, enzymes (HRP), nucleic acid amplification primers (RCA, PCR, LAMP, etc.) or iii) drug delivery such as small molecule drugs, antimicrobial agents, and gene therapeutics. In certain embodiments, the 3′ arm comprises another aptamer (‘B’) that may be identical or different to the ‘A’ aptamer element. In certain embodiments, for example for reporter constructs, the ‘B’ element may possess a fluorescent reporter molecule, antigen, or enzyme. In other embodiments, for example for immobilization constructs, the ‘B’ element may possess functional molecules to enable crosslinking such as thiol, amide, biotin, digoxigenin, azide, alkyne, carboxyl, or crosslinkers compatible with Click-Chemistry methodologies.

### Aptamer Elements (A)

According to embodiments, the individual ‘A’ units or elements are aptamers specific for one or more epitopes of a target molecule or protein. Aptamer elements (A) suitable for the multivalent trident aptamer construct, may be aptamers specific for epitopes of a target molecule or protein associated with a disease or disorder. A wide variety of aptamers that are specific to such target molecules or proteins are known in the art. Appropriate aptamers can be readily selected by one skilled in the art based on, for example, the desired end use of the multivalent trident aptamer, such as the disease or disorder against which it is to be directed and/or the subject to which it is to be administered.

For example, the aptamer can be specific for a target molecule or protein associated with a disease or disorder in an animal, such as a cancer, infectious disease, allergic reaction, or autoimmune disease. The aptamer may be specific for one or more epitopes associated with a pathogen known in the art, such as, for example, a bacterium, virus, protozoan, fungus, parasite, or infectious particle, such as a prion, or it may be a tumour-associated epitope or other biomarker.

According to embodiments, aptamers specific for one or more epitopes associated with viral targets include, for example, aptamers specific for epitopes associated with members of the families Adenoviradae; Arenaviridae (for example, Ippy virus and Lassa virus); Birnaviridae; Bunyaviridae; Caliciviridae; Coronaviridae; Filoviridae; Flaviviridae (for example, yellow fever virus, dengue fever virus and hepatitis C virus); Hepadnaviradae (for example, hepatitis B virus); Herpesviradae (for example, human herpes simplex virus 1); Orthomyxoviridae (for example, influenza virus A, B and C); Paramyxoviridae (for example, mumps virus, measles virus and respiratory syncytial virus); Picornaviridae (for example, poliovirus and hepatitis A virus); Poxyiridae; Reoviridae; Retroviradae (for example, BLV-HTLV retrovirus, HIV-1, HIV-2, bovine immunodeficiency virus and feline immunodeficiency virus); Rhabodoviridae (for example, rabies virus), and Togaviridae (for example, rubella virus).

In one embodiment, the multivalent trident aptamer comprises one or more aptamers (A) specific for the target molecule or protein associated with a major viral pathogen such as the dengue virus, various hepatitis viruses, human immunodeficiency virus (HIV), various influenza viruses, West Nile virus, respiratory syncytial virus, influenza virus, rabies virus, human papilloma virus (HPV), Epstein Barr virus (EBV), polyoma virus, or SARS coronavirus.

According to certain embodiments, the multivalent trident aptamer comprises one or more aptamers (A) specific for the target molecule SARS-COV-2 S protein. In further embodiments, the multivalent trident aptamer comprises one or more MSA52 aptamers. In other embodiments, the multivalent trident aptamer comprises one or more MSA52T8 aptamers. In other embodiments, the multivalent trident aptamer comprises one or more aptamers (A) specific for the target molecule Influenza HA protein. In further embodiments, the multivalent trident aptamer comprises one or more RHA06 aptamers.

According to embodiments, aptamers specific for one or more epitopes associated with bacterial targets include, for example, aptamers specific for epitopes associated with known causative agents responsible for diseases such as dyptheria, pertussis, tetanus, tuberculosis, bacterial pneumonia, fungal pneumonia, cholera, typhoid, plague, shigellosis, salmonellosis, Legionnaire's disease, Lyme disease, leprosy, malaria, hookworm, onchocerciasis, schistosomiasis, trypanosomiasis, Leishhmaniasis, giardia, amoebiasis, filariasis, borrelia, and trichinosis.

According to embodiments, aptamers specific for one or more epitopes associated with tumour-associated targets include, for example, aptamers specific for epitopes associated with HER2 (breast cancer); GD2 (neuroblastoma); EGF-R (malignant glioblastoma); CEA (medullary thyroid cancer); CD52 (leukemia); human melanoma protein gp100; human melanoma protein melan-A/MART-1; NA17-A nt protein; p53 protein; various MAGEs (melanoma associated antigen E), including MAGE 1, MAGE 2, MAGE 3 (HLA-A1 peptide) and MAGE 4; various tyrosinases (HLA-A2 peptide); mutant ras; p97 melanoma antigen; Ras peptide and p53 peptide associated with advanced cancers; the HPV 16/18 and E6/E7 antigens associated with cervical cancers; MUC1-KLH antigen associated with breast carcinoma; CEA (carcinoembryonic antigen) associated with colorectal cancer, DKK-1 (Dickkopf-1 protein) associated with lung cancer and the PSA associated with prostate cancer.

According to certain embodiments, the multivalent trident aptamer comprises one or more aptamers (A) specific for the target molecule VEGF165 protein. In further embodiments, the multivalent trident aptamer comprises one or more H1A aptamers.

According to other embodiments, the multivalent trident aptamer comprises one or more aptamers (A) specific for a biomarker that is predictive, prognostic, or diagnostic of a clinical condition. In certain embodiments, the multivalent trident aptamer comprises one or more aptamers (A) specific for the target molecule Troponin I protein. In further embodiments, the multivalent trident aptamer comprises one or more TnAp1 aptamers.

According to certain embodiments, the multivalent trident aptamer comprises one or more aptamers (A) specific for the target molecule SARS-COV-2 spike protein, Influenza hemagglutinin (HA) and neuraminidase (NA) proteins, Alkaline Phosphatase, other VEGF family proteins (VEGF121, VEGF145, VEGF-A, PGF), Platelet-derived growth factor (PDGF), norovirus capsid protein (VP1).

In certain embodiments, the aptamer (A) element may be up to 150 nucleotides in length. In other embodiments, the aptamer (A) element may be from 5 to 150 nucleotides in length. In further embodiments, the aptamer (A) element may be 10 to 100 nucleotides in length. In other embodiments the aptamer (A) element may be 15 to 90 nucleotides in length. In further embodiments, the aptamer (A) element may be 20 to 90 nucleotides in length.

In some embodiments, the multivalent trident aptamer is a homo-trimeric aptamer trident construct where each of the trident arms/branches comprise an identical aptamer element (A). According to other embodiments, the multivalent trident aptamer is a hetero-trimeric aptamer trident construct where two of the three trident arms/branches comprise an identical aptamer element (A). In a further embodiment, the multivalent trident aptamer is a hetero-trimeric aptamer trident construct where each of the trident arms/branches comprise an aptamer element (A) that is specific to a different epitope of a target molecule or protein.

In some embodiments, the one or more aptamer element (A) is modified to be more resistant to denaturation according to methods known in the art. For example, aptamer elements (A) may be modified to include a locked nucleic acid (LNA), 2′-O-methyl nucleotides, or 2′-fluoro-deoxyribonucleotides. In certain embodiments, the one or more aptamer element (A) is modified to comprise a locked nucleic acid (LNA), 2′-O-methyl nucleotides, or 2′-fluoro-deoxyribonucleotides.

### Spacers (‘S [A,B]’)

Each aptamer element (A), according to embodiments, will interact with target epitopes via non-covalent forces (i.e. hydrogen bonding, hydrophobic interactions, and electrostatic interactions) based on the unique three-dimensional structure of the aptamer element (A). Alternatively, binding elements may interact with target proteins via sequence specific nucleic acid binding properties of the target protein.

According to embodiments, the spacing of the aptamer element (A) from the target epitope is optimized to allow sufficient reach such that each aptamer (A) can reach its respective epitope on the target, without being excessively long such that aptamer folding is inhibited or steric inhibition is observed. In certain embodiments, for example, binding clusters of epitopes on a target is desired, and in such embodiments, the spacing of the aptamer (A) is restricted to limit binding to a local area (i.e., a single target molecule on a cell surface with multiple targets). According to further embodiments, the spacer molecule (S_{A}) affords flexibility to allow for a fluctuation of aptamer (A) distance to cover the distance ranges of the target molecule.

In certain embodiments, the S_{A} is a spacer molecule that separates an aptamer (A) element from the respective terminal end of the L-branch at a distance of up to 25 nm. In other embodiments, the spacer (S_{A}) has a span of up to 25 nm, 20.5 nm, 18 nm, 15 nm, 12 nm, 10 nm, 8 nm, 5 nm, or 0 nm. In other embodiments, the spacer (S_{A}) has a span of 5 nm to 25 nm, 10 nm to 20.5 nm, or 10 nm to 18 nm. In some embodiments, the spacer S_{A} has the same span length for each aptamer (A) on its respective terminal end of the L-branch. In other embodiments, the span length of each spacer S_{A} is different for each aptamer (A)-L- branch.

According to embodiments, the spacer (S_{A}) is a linear carbon molecule. In such embodiments, the linear carbon molecule comprises up to 15 linear alkane chains each possessing up to 12 carbon atoms. In other embodiments, the spacer (S_{A}) is a linear polyethylene glycol chain possessing up to 55 ethylene glycol units. In further embodiments, the spacer (S_{A}) is an unstructured flexible single stranded nucleic acid sequence of up to 30 deoxythymidine or other bases. In other embodiments, the spacer (S_{A}) is an unstructured flexible single stranded nucleic acid sequence of 10 to 30, 15 to 30, 20 to 30, or 25 to 30 deoxythymidine or other bases.

According to some embodiments, the root branch of the multivalent trident aptamer may comprise a spacer (S_{B}). In some embodiments, the S_{B} spacer molecule has a span of up to 25 nm, 20.5 nm, 18 nm, 15 nm, 12 nm, 10 nm, 8 nm, 5 nm, or 0 nm. In other embodiments, the spacer (S_{B}) has a span of 5 nm to 25 nm, 10 nm to 20.5 nm, or 10 nm to 18 nm. In some embodiments, the spacer S_{B} has the same span length as each S_{A} between an aptamer (A) and its respective terminal end of the L-branch. According to embodiments, the spacer (S_{B}) is a linear carbon molecule. In such embodiments, the linear carbon molecule comprises up to 15 linear alkane chains each possessing up to 12 carbon atoms. In other embodiments, the spacer (S_{B}) is a linear polyethylene glycol chain possessing up to 55 ethylene glycol units. In further embodiments, the spacer (S_{B}) is an unstructured flexible single stranded nucleic acid sequence of up to 30 deoxythymidine or other bases. In other embodiments, the spacer (S_{B}) is an unstructured flexible single stranded nucleic acid sequence of 10 to 30, 15 to 30, 20 to 30, or 25 to 30 deoxythymidine or other bases.

Persons of skill in the art will readily appreciate the spacers that can be used. For example, in certain embodiments, the spacer (S_{A} and/or (the spacer (S_{B}) is Spacer Phosphoramidite 18 (https://www.glenresearch.com/spacer-modifiers/10-1918.html) or Spacer C12 CE Phosphoramidite (https://www.glenresearch.com/spacer-modifiers/10-1928.html).

### Linkers (‘L’)

According to embodiments, the multivalent trident aptamer comprises a linker (L) having up to three variable branches/arms and a root, wherein each branch comprises a terminal end that links to one of the [A-S_{A}]s to form a trident configuration. The linker (‘L’) is a linker molecule connecting ‘A’ and ‘B’ elements via optional spacers S [A,B]. According to embodiments, the linker molecule (L) may be a doubler or trebler or another branching chemical group with multiple arms/branches.

According to certain embodiments, the linker ‘L’ is a trebler phosphoramidite. In other embodiments, the linker (L) is a doubler phosphoramidite. In certain embodiments, the multivalent trident aptamer is a symmetrical trident construct. In such embodiments, the linker molecule (L) is a Symmetric Trebler molecule (Glen Research 10-1922).

In other embodiments, the multivalent trident aptamer is an asymmetrical trident construct. In such embodiments, the linker molecule (L) may be composed of two sequential units of Asymmetric Doubler molecule (Glen Research 10-1981).

In further embodiments, the multivalent trident aptamer is a symmetric fork construct. In such embodiments, the linker molecule (L) may be a Symmetric Doubler molecule (Glen Research 10-1920). In further embodiments, the multivalent trident aptamer is a symmetric fork construct. In other embodiments, the linker molecule (L) may be an asymmetric doubler construct. In such embodiments, the linker molecule (L) may be an Asymmetric Doubler molecule (Glen Research 10-1981).

Further embodiments provide for orientation control. Given that aptamers and their targets are known to bind in a preferred relative orientation, the multivalent trident aptamer constructs allow for the attachment of homomeric or heteromeric aptamers in either the 5′-3′ or 3′-5′ orientation relative to the central linker molecule (L). This allows for the aptamers to be arranged in an optimal orientation for epitope binding. According to embodiments, the aptamer element (A) may be connected to the linker (L) from either the 5′, 3′ or at an internal position of the aptamer sequence. In other embodiments, the multivalent trident aptamer constructs comprise aptamers (A) that are in the same 5′ to 3′ orientation relative to the linker (L). In further embodiments, the multivalent trident aptamer constructs comprise aptamers (A) that are in the same 3′ to 5′ orientation relative to the linker (L).

### 3′ Branch or Root [B]

According to embodiments, the 3′ branch or root of the multivalent trident aptamer construct provides a multipurpose attachment site on the 3′ branch/root of the trebler or doubler linker. In such embodiments, the 3′ root may be functionalized with a variety of other molecules to enable i) surface immobilization or crosslinking via biotin, amine, thiol or digoxigenin molecules, ii) attachment of reporter elements such as fluorophores, enzymes (HRP), nucleic acid amplification primers (RCA, PCR, LAMP, etc.) or iii) drug delivery such as small molecule drugs, antimicrobial agents, and gene therapeutics.

In certain embodiments, the 3′ root of the multivalent trident aptamer is attached to a functional molecule (B) selected from an aptamer, a reporter molecule, or a crosslinker. In certain embodiments, the functional molecule (B) is an aptamer identical to one or more of the individual aptamers (A). In other embodiments, the functional molecule (B) is a reporter molecule selected from an antigen, an enzyme, and a fluorescent molecule. In further embodiments, the functional molecule (B) is a crosslinker selected from thiol, amide, biotin, digoxigenein, azide, alkyne, carboxyl, and a click-chemistry-based crosslinker.

### Binding Affinity & Avidity

In various embodiments, the multivalent trident aptamer described herein, and/or compositions or formulations comprising these trident aptamers, exhibit enhanced affinity and/or avidity for a target molecule. According to certain embodiments, the multivalent trident aptamers described herein exhibit a synergistic binding affinity and/or avidity to a target. In particular, it was unexpectedly found that the multivalent trident aptamers described herein exhibit an enhanced or synergistic affinity/avidity for a target molecule of up to 80-fold over its corresponding monomeric or dimeric form. The unexpectedly enhanced affinity/avidity of the trident aptamer was observed in its binding with target molecules of various forms, including monomeric, dimeric, and trimeric protein targets. Moreover, the trident construct displayed cooperativity between each branch in binding the target molecule.

In certain embodiments, the multivalent trident aptamers described herein cooperatively bind to a trimeric protein target. In other embodiments, the multivalent trident aptamers described herein cooperatively bind to a dimeric protein target. In further embodiments, the multivalent trident aptamers described herein cooperatively bind to a monomeric protein target.

According to certain embodiments, the multivalent trident aptamers described herein exhibit up to an 80-fold improvement in affinity/avidity with its target molecule over the affinity of each individual aptamer (A). In further embodiments, the multivalent trident aptamers described herein exhibit up to a 75-fold improvement in affinity/avidity with its target molecule over the affinity of each individual aptamer (A). According to other embodiments, the multivalent trident aptamers described herein exhibit up to a 50-fold improvement in affinity/avidity with its target molecule over the affinity of each individual aptamer (A). According to other embodiments, the multivalent trident aptamers described herein exhibit up to a 40-fold improvement in affinity/avidity with its target molecule over the affinity of each individual aptamer (A). According to certain embodiments, the multivalent trident aptamers described herein exhibit up to a 30-fold improvement in affinity/avidity with its target molecule over the affinity of each individual aptamer (A).

### Methods and Uses

The enhanced, cooperative binding affinity of the multivalent trident aptamer constructs described herein offers a range of applications. Moreover, the multivalent trident aptamer construct provides a multipurpose attachment site on the 3′ branch of the linker ([S_{B}-B]) to allow for versatile functionalization with a variety of other molecules to enable i) surface immobilization or crosslinking via biotin, amine, thiol or digoxigenin molecules, for example, ii) attachment of reporter elements such as fluorophores, enzymes (HRP), nucleic acid amplification primers (RCA, PCR, LAMP, etc.) or iii) drug delivery such as small molecule drugs, antimicrobial agents, gene therapeutics.

In certain embodiments, the 3′ branch can comprise another aptamer (‘B’) that may be identical or different to the ‘A’ aptamer element. In certain embodiments, for example for reporter constructs, the ‘B’ element may possess a fluorescent reporter molecule, antigen or enzyme. In other embodiments, for example for immobilization constructs, the ‘B’ element may possess functional molecules to enable crosslinking such as thiol, amide, biotin, digoxigenin, azide, alkyne, carboxyl or crosslinkers compatible with Click-Chemistry methodologies.

### Methods of Treatment

The multivalent trident aptamers of the present disclosure are useful in a variety of applications including, but not limited to, methods for the treatment of a disorder or disease. In particular, the multivalent trident aptamers described herein provide a method for targeted drug delivery to one or more targets, comprising administering to a cell, or a subject, trident aptamers of the invention that exhibits a therapeutic effect, or that is loaded with a drug via the 3′ branch of the trident aptamer, to target the cell or tissue for treatment. In other embodiments, the multivalent trident aptamers described herein provide a method for targeted binding and neutralization of a pathogen.

Further provided are pharmaceutical compositions, comprising a multivalent trident aptamer of the invention and one or more pharmaceutically acceptable excipients. In certain embodiments, the present invention provides for pharmaceutical compositions comprising an effective amount of a multivalent trident aptamer and one or more pharmaceutically acceptable carriers, diluents and/or excipients. If desired, other active ingredients may be included in the compositions, for example, additional immune stimulating compounds, standard therapeutics, vaccines or the like.

The pharmaceutical compositions can be formulated for administration by a variety of routes. For example, the compositions can be formulated for oral, topical, rectal, nasal, or parenteral administration or for administration by inhalation or spray. The term parenteral as used herein includes subcutaneous injections, intravenous, intramuscular, intrathecal, intrasternal injection or infusion techniques. Intranasal administration to the subject includes administering the composition to the mucous membranes of the nasal passage or nasal cavity of the subject.

In some embodiments, the pharmaceutical compositions are formulated for mucosal administration. Mucosal administration may include, for example, oral, intranasal, aerosol, rectal, or vaginal administration. The preparations for mucosal administration include transdermal devices, aerosols, creams, lotions, or powders pending on the mucosal site. In certain embodiments, the pharmaceutical compositions are formulated for intranasal or pulmonary administration. In some embodiments, the pharmaceutical compositions are formulated for rectal or vaginal administration.

Compositions formulated as aqueous suspensions may contain the multivalent trident aptamer in admixture with one or more suitable excipients, for example, with suspending agents, such as sodium carboxymethylcellulose, methyl cellulose, hydroxypropylmethylcellulose, sodium alginate, polyvinylpyrrolidone, hydroxypropyl-β-cyclodextrin, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyoxyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

In certain embodiments, the pharmaceutical compositions may be formulated as oily suspensions by suspending the drug-loaded multivalent trident aptamer in a vegetable oil, for example, arachis oil, olive oil, sesame oil or coconut oil, or in a mineral oil such as liquid paraffin. The oily suspensions may contain a thickening agent, for example, beeswax, hard paraffin, or cetyl alcohol. These compositions can be preserved by the addition of an antioxidant such as ascorbic acid.

In certain embodiments, the pharmaceutical compositions may be formulated as a dispersible powder or granules, which can subsequently be used to prepare an aqueous suspension by the addition of water. Such dispersible powders or granules provide the multivalent trident aptamer in admixture with one or more dispersing or wetting agents, suspending agents and/or preservatives. Suitable dispersing or wetting agents and suspending agents are exemplified by those already mentioned above. Additional excipients, for example, colouring agents, can also be included in these compositions.

Pharmaceutical compositions of the invention may also be formulated as oil-in-water emulsions in some embodiments. The oil phase can be a vegetable oil, for example, olive oil or arachis oil, or a mineral oil, for example, liquid paraffin, or it may be a mixture of these oils. Suitable emulsifying agents for inclusion in these compositions include naturally occurring gums, for example, gum acacia or gum tragacanth; naturally occurring phosphatides, for example, soy bean, lecithin, or esters or partial esters derived from fatty acids and hexitol, anhydrides, for example, sorbitan monoleate, and condensation products of the said partial esters with ethylene oxide, for example, polyoxyethylene sorbitan monooleate.

In certain embodiments, the pharmaceutical compositions may be formulated as a sterile injectable aqueous or oleaginous suspension according to methods known in the art and using suitable one or more dispersing or wetting agents and/or suspending agents, such as those mentioned above. The sterile injectable preparation can be a sterile injectable solution or suspension in a non-toxic parentally acceptable diluent or solvent, for example, as a solution in 1,3-butanediol. Acceptable vehicles and solvents that can be employed include, but are not limited to, water, Ringer's solution, lactated Ringer's solution, and isotonic sodium chloride solution. Other examples include, sterile, fixed oils, which are conventionally employed as a solvent or suspending medium, and a variety of bland fixed oils including, for example, synthetic mono- or diglycerides. Fatty acids such as oleic acid can also be used in the preparation of injectables.

Optionally the pharmaceutical compositions may contain preservatives such as antimicrobial agents, antioxidants, chelating agents, and inert gases, and/or stabilizers such as a carbohydrate (e.g. sorbitol, mannitol, starch, sucrose, glucose, or dextran), a protein (e.g. albumin or casein), or a protein-containing agent (e.g. bovine serum or skimmed milk) together with a suitable buffer (e.g. phosphate buffer). The pH and exact concentration of the various components of the composition may be adjusted according to well-known parameters.

Sterile compositions can be prepared for example by incorporating the multivalent trident aptamer in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile compositions, some exemplary methods of preparation are vacuum-drying and freeze-drying techniques which yield a powder of the active ingredient plus any additional desired ingredient from a previously sterile-filtered solution thereof.

Contemplated for use in certain embodiments of the invention are various mechanical devices designed for pulmonary or intranasal delivery of therapeutic products, including but not limited to, nebulizers, metered dose inhalers, powder inhalers and nasal spray devices, all of which are familiar to those skilled in the art.

Metered dose inhalers typically use a propellant gas and require actuation during inspiration. Dry powder inhalers use breath-actuation of a mixed powder. Nebulizers produce aerosols from solutions, while metered dose inhalers, dry powder inhalers, and the like generate small particle aerosols.

Some specific examples of commercially available mechanical devices include the ULTRAVENT® nebulizer (Mallinckrodt, Inc., St. Louis, Mo.), the ACORN II® nebulizer (Marquest Medical Products, Englewood, Colo.), the MISTY-NEB® nebulizer (Allegiance, McGraw Park, Ill.), the AEROECLIPSE® nebulizer (Trudell Medical International, Canada), the Accuspray™ nasal spray device (Becton Dickinson), the Mucosal Atomization Device (MAD300) (Wolfe Tory Medical), the OptiNose device (OptiNose, Oslo, Norway), the Nektar DPI system (Nektar Therapeutics, Inc., San Carlos, Calif.), the AERx pulmonary drug delivery system (Aradigm Corporation, Hayward, Calif.), the Spiros® device (Dura Pharmaceuticals), and the Respimat® device (Boehringer Ingelheim).

All such devices require the use of formulations suitable for the dispensing of the multivalent trident aptamer. Typically, each formulation is specific to the type of device employed and may involve the use of an appropriate propellant material, in addition to the usual diluents, adjuvants and/or carriers useful in therapy as would be understood by a worker skilled in the art. Also, the use of liposomes, microcapsules or microspheres, inclusion complexes, or other types of carriers is contemplated.

Other pharmaceutical compositions and methods of preparing pharmaceutical compositions are known in the art and are described, for example, in “Remington: The Science and Practice of Pharmacy” (formerly “Remington Pharmaceutical Sciences”); Gennaro, A., Lippincott, Williams & Wilkins, Philadelphia, Pa. (2000).

### Methods of Detection and Diagnosis

The multivalent trident aptamers of the present disclosure are useful in a variety of applications including, but not limited to, methods for detecting the presence of a target molecule. In one aspect, the multivalent trident aptamers are useful for detecting the presence of a target molecule in a biological sample. The term “detecting” as used herein includes quantitative or qualitative detection.

In one aspect, the present disclosure provides a method of detecting the presence of a target molecule in a biological sample. In certain aspects, the method comprises contacting the biological sample with a multivalent trident aptamer specific for the target molecule under conditions permissive for binding of the multivalent trident aptamer and detecting whether a complex is formed between the multivalent trident aptamer and the target. The biological sample can include, without limitation, urine or blood samples.

In some embodiments, detection of the target molecule is indicated by a fluorescent, colorimetric, electrochemical, surface plasmon resonance, spectroscopic, or radioactive signal. In some embodiments, detection of the target molecule is indicated by a fluorescent signal. In some embodiments, an increase in the fluorescence signal indicates presence of the target in the sample.

### Kits

In certain aspects of the invention, kits are provided comprising a container housing a composition comprising the multivalent trident aptamers.

### Pharmaceutical Kits

Certain embodiments of the invention provide for pharmaceutical kits comprising multivalent trident aptamers for use as a therapeutic. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the multivalent trident aptamers.

When the kit comprises multivalent trident aptamers for use as a drug delivery system, the kit may further comprise one or more drugs for use in combination with the multivalent trident aptamers.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, nasal spray device, nebulizer, syringe, pipette, forceps, measured spoon, eye dropper or similar medically approved delivery vehicle.

### Target Detection Kits

According to certain embodiments, the multivalent trident aptamer can be combined into a test kit system. For example, the multivalent trident aptamer can be combined into a biosensor system or kit for use to detect any suitable target analyte, such as, and without being limited thereto, a wide range of small molecule, protein, and nucleic acid analytes, including infection-causing pathogens in point-of-care testing for screening, diagnostics and/or health monitoring.

In some embodiments, the sample is a biological sample, and the presence of the target in the sample is indicative of, or associated, with a disease, disorder, or condition. In some embodiments, the target is a pathogen. Accordingly, provided is a method of detecting a pathogen infection in a subject comprising testing a sample from the subject for the presence of a target using the multivalent trident aptamer combined into a biosensor, biosensor system and/or kit, wherein presence of a target indicates that the subject has an infection.

In accordance with another aspect, there is provided a kit for detection of a target in a sample comprising the multivalent trident aptamer combined into a biosensor or biosensor system and instructions for use.

To gain a better understanding of the invention described herein, the following examples are set forth. It will be understood that these examples are intended to describe illustrative embodiments of the invention and are not intended to limit the scope of the invention in any way.

### EXAMPLES

### Example 1: Preparation of Multivalent Trident Aptamer

Chemical Synthesis of Trident Aptamers. The synthesis of trident aptamers was carried out using an automated oligonucleotide synthesizer, specifically the Mermade 12 Synthesizer from Biosearch process followed the manufacturer's instructions and utilized the recommended reagents and default coupling times, unless specified otherwise by Glen Research. The synthesis began by coupling the immobilization modifications, Thiol-Modifier C6 S—S or Protected Biotin Serinol Phosphoramidite, to the 3′ end of the strand. This step was performed on Glen UnySupport 1400 CPG at either 1 μmol or 5 μmol synthesis scales. Subsequently, the branched linker, Trebler phosphoramidite, was coupled. The aptamer sequence was then coupled in the 3′ to 5′ direction following the linker. To ensure successful couplings after the addition of Trebler, the concentrations of dA, dC, dG, and dT phosphoramidites were doubled from the recommended Glen Research concentrations. Upon completion of the synthesis, the oligos were cleaved from the CPG support and deprotected using a 1:1 mixture of ammonium hydroxide and methylamine, following the UltraFast deprotection method provided by Glen Research (source: https://www.glenresearch.com/reports/gr26-14). The deprotected oligos were dried using a Speed Vac, dissolved in 1×LB buffer, and heated at 90° C. for 2 minutes to denature the sample. Subsequently, the samples were purified using a 7.5% 8 M urea polyacrylamide gel electrophoresis (PAGE) gel to separate the full-length synthesis products. Gel fragments containing the desired full-length products were excised, crushed, and eluted in elution buffer (EB). The EB supernatant was collected, and ethanol precipitation was performed using 2.5 volumes of cold ethanol, followed by a wash with 70% cold ethanol. The precipitated oligonucleotides were pelleted by centrifugation at a speed exceeding 20,000 rcf for 20 minutes. After removing the supernatant, the pellets were dried and resuspended in nuclease-free water. Finally, the gel-purified oligonucleotides were quantified spectroscopically by measuring the absorbance at 260 nm, allowing for accurate determination of their concentration.

### Exemplary Trident Aptamers for Binding Influenza HA, VEGF165, Troponin-I and SARS-COV-2 S-Proteins.

To evaluate the versatility of trident aptamers, four disease-related protein biomarkers were selected as aptamer binding targets: influenza hemagglutinin (HA) protein, SARS-COV-2 spike(S) protein, vascular endothelial growth factor 165 (VEGF165, a cancer biomarker) protein, and troponin-I protein (a cardiac biomarker). For each target protein, one monomeric and one trident aptamer, resulting in a total of eight aptamers were prepared for testing. The DNA sequences used in the preparation of these aptamers are presented in Table 1. Additionally, two mutant DNA controls with random sequences and a protein control (bovine serum albumin) were also prepared.

### Influenza Hemagglutinin (HA) Protein

A symmetric trident RHA06 aptamer was synthesized to target influenza HA, because the RHA06 aptamer was confirmed to bind several variant HA proteins, including influenza H1N1, H3N2, and H5N1. The design of the trident RHA06 aptamer was based on the size of the HA protein. HA protein has a diameter of 7-14 nm, with a spacing of 10 nm between each monomer center. Each branch of the trident RHA06, containing 60 nucleotides, is around 20 nm. Due to the flexibility of the process, the trident RHA06 should allow for a fluctuation in distance from 0 to 34 nm. This range completely covers the distance range between each subunit of the trimeric HA protein, providing enough flexibility for the trident RHA06 to bind to the trimeric HA.

### VEGE, Troponin-I, and SARS-COV-2 S1 Subunit Proteins

In addition to influenza HA and SARS-COV-2 Protein, trident aptamers for three other biomarker proteins: VEGF, troponin-I, and SARS-COV-2 S1 subunit proteins were prepared using the same synthesis method.

TABLE 1
Sequences of DNA oligonucleotides.

| | | Size | Sequence (5′-3′) |
|---|---|---|---|
| Name | Target | (nt) | italic nucleotides act as linkers |
| MSA52T8 | SARS-CoV- | 49 | CGAAGCATCTCGTGTCACTCCGTAGGGTTTGGCTCCGGGCCTGGCGTCG |
| | 2 S | | |
| TMSA52T8 | SARS-CoV- | 188 | CGAAGCATCTCGTGTCACTCCGTAGGGTTTGGCTCCGGGCCTGGCGTCG |
| | 2 S | | TTTTTCTCTCGC-Trebler-TTTTT-Biotin |
| Mutant- | | 188 | CGAAGCATCTCGTGTCACTCGAGCTGGTTTGGCGGCCTGCCTGGCGTCG |
| Trimer | | | TTTTTCTCTCGC-Trebler-TTTTT-Biotin |
| RHA06 | Influenza | 48 | GGGTTTGGGTTGGGTTGGGTTTTTGGGTTTGGGTTGGGTTGGGAAAAA |
| | HA | | |
| Trimeric | Influenza | 185 | GGGTTTGGGTTGGGTTGGGTTTTTGGGTTTGGGTTGGGTTGGGAAAAAT |
| RHA06 | HA | | TTTTCTCTCGC-Trebler-TTTTT-Biotin |
| Mutant- | | 185 | GTTTGGTTATGGGAGTGTGGGGGGAGGTTGGTGGTGTGTGGTTATTATT |
| Trimer | | | TTTTCTCTCGC-Trebler-TTTTT-Biotin |
| H1A | VEGF165 | 28 | GCCCGTCTTCCAGACAAGAGTGCAGGGC |
| Trimeric | VEGF165 | 125 | GCCCGTCTTCCAGACAAGAGTGCAGGGCTTTTTCTCTCGC-Trebler- |
| H1A | | | TTTTT-Biotin |
| TnAp1 | Troponin | 80 | GGCAGCAGGAAGACAAGACATGGGTGGCGGGGACGGGGCGATGGGAACT |
| | I | | TAGATTGCTAGTGGTTCTGTGGTTGCTCTGT |
| Trimeric | Troponin | 281 | GGCAGCAGGAAGACAAGACATGGGTGGCGGGGACGGGGCGATGGGAACT |
| TnAp1 | I | | TAGATTGCTAGTGGTTCTGTGGTTGCTCTGTTTTTTCTCTCGC- |
| | | | Trebler-TTTTT-Biotin |

### Example 2: Assessment of Binding Affinity

The binding affinity of the synthesized monomeric and trident aptamers were assessed using the standard dot blot assays, a technique that has been widely used to determine the affinity of protein binding aptamers. Dot blot assays involve the use of two distinct membranes: nitrocellulose and nylon to separate the bound aptamers with proteins from the unbound aptamers. The aptamers were labeled with ³²P to generate a radioactive signal, allowing the calculation of the fraction of bound aptamers to proteins. By fitting the fraction of bound aptamers, the dissociation constants (Kd values) of the assessed aptamers can be derived. The Kd values were then used to determine and compare the strength of the binding interaction between the aptamers and target proteins. A lower Kd value indicates a higher affinity, meaning that the binding between aptamers and proteins is stronger and less likely to dissociate.

Dot Blot Binding Assays. Dot blot assays were performed by using a Whatman Minifold-1 96-well apparatus and a vacuum pump. Before experiments, nitrocellulose membranes and nylon membranes were incubated in 1× binding buffer for 1 h. γ-[³²P] labelled DNA aptamers (1 nM) were dissolved in the binding buffer and heated at 90° C. for 5 min, and then cooled at room temperature for 20 min. Proteins were dissolved and diluted in the same buffer. 5 μL of the above aptamer solution was mixed with 15 μL of protein with different concentrations. The mixture was incubated at room temperature for 1 h. The dot blot apparatus was assembled with a nitrocellulose membrane on the top, a nylon membrane in the middle and a wetted Whatman paper in the bottom. After washing each well with 100 μL of binding buffer, the binding mixtures were loaded and drained by the vacuum pump (force: 550 mmHg for 8 seconds). The wells were then washed twice with 100 μL binding buffer. The membranes were imaged using a Typhoon 9200 imager (GE Healthcare) and analyzed using Image J software. Each binding assay was performed 3 times. The bound fraction was quantified and plotted against the concentration of the protein. The K_{d} values were derived via curve fitting using Origin 8.0 using the equation Y=BₘₐₓX/(K_{d}+X) (Y is the bound fraction of aptamer with protein, Bₘₐₓ is the maximum bound fraction of aptamer, and X is protein concentration).

Radiolabelling of DNA Aptamers. DNA aptamers were labeled with γ-[³²P] ATP at the 5′-end using PNK reactions according to the manufacturer's protocol. Briefly, 2 μL of 1 μM DNA aptamers were mixed with 2 μL of γ-[³²P] ATP, 1 μL of 10×PNK reaction buffer A, 10 U (U: unit) of PNK and 4 μL water. The mixture was incubated at 37° C. for 20 min, and then purified by 10% dPAGE.

### Influenza Hemagglutinin (HA) Protein

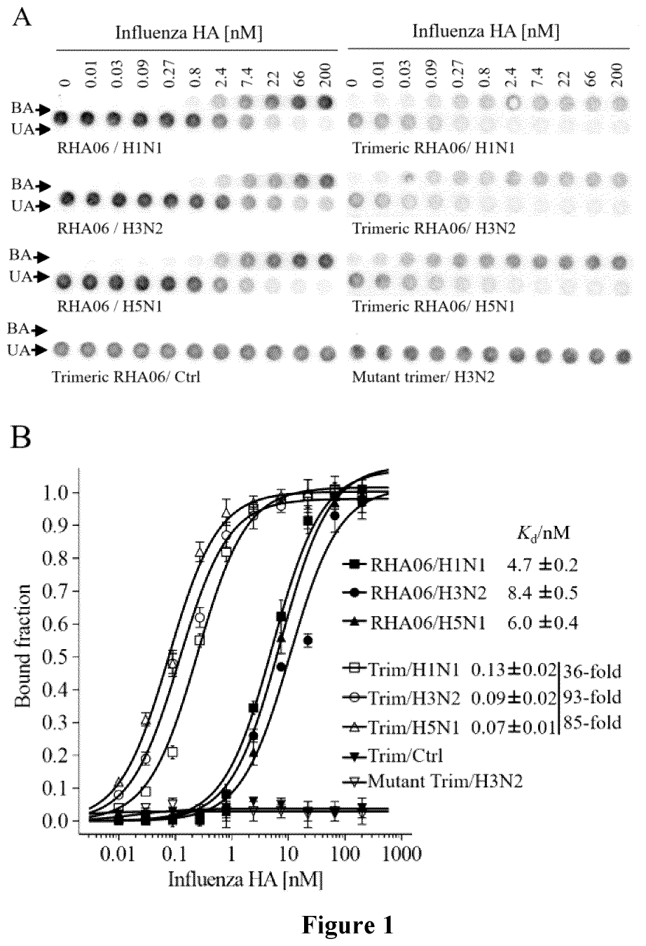

The affinity of RHA06 for the variant HA proteins was assessed using dot blot assays. The results of the dot blots are provided in FIG. 1A, and the derived Kd values are shown in FIG. 1B. RHA06 effectively bound to the three variant HA proteins, with similar Kd values ranging from 4.7 to 8.4 nM, confirming its universal ability to bind variant influenza HA.

However, nanomolar level affinity is not sensitive enough for detecting influenza in biological samples due to the low loading of detectable targets in clinical samples. Considering that HA protein is a symmetric trimeric protein, we hypothesized that constructing a symmetric aptamer capable of forming a three-to-three aptamer-protein complex could improve the affinity. The effectiveness of this approach was previously demonstrated for binding the trimeric S-protein of SARS-COV-2, where the affinity for binding the S-protein reached the picomolar level (U.S. Patent Application No. 63/409,455). Therefore, the same method was used to synthesize the symmetric trident RHA06 aptamer.

After assessing the affinity of the monomeric RHA06 for variant HA proteins, the affinity of the constructed trident RHA06 aptamer was assessed using the same dot blot assays. The dot blot results, and derived Kd values are provided in FIG. 1 for comparison with the monomeric RHA06. The trident RHA06 demonstrated significantly enhanced affinities, with Kd values of 0.07, 0.09, and 0.13 nM for the H1N1, H3N2, and H5N1-HA proteins, respectively. These affinities were 36-, 93-, and 85-fold higher than those of the monomeric RHA06 aptamer for the same proteins. These results confirmed that the trident aptamer approach can indeed enhance aptamer binding affinity, likely due to the symmetric formation between the trident RHA06 and trimeric HA proteins. The trident RHA06 showed no binding to the control protein BSA or the mutant trimeric DNA with H3N2-HA protein, indicating the specificity of the trident RHA06 for influenza HA.

### VEGE, Troponin-I, and SARS-COV-2 S1 Subunit Proteins

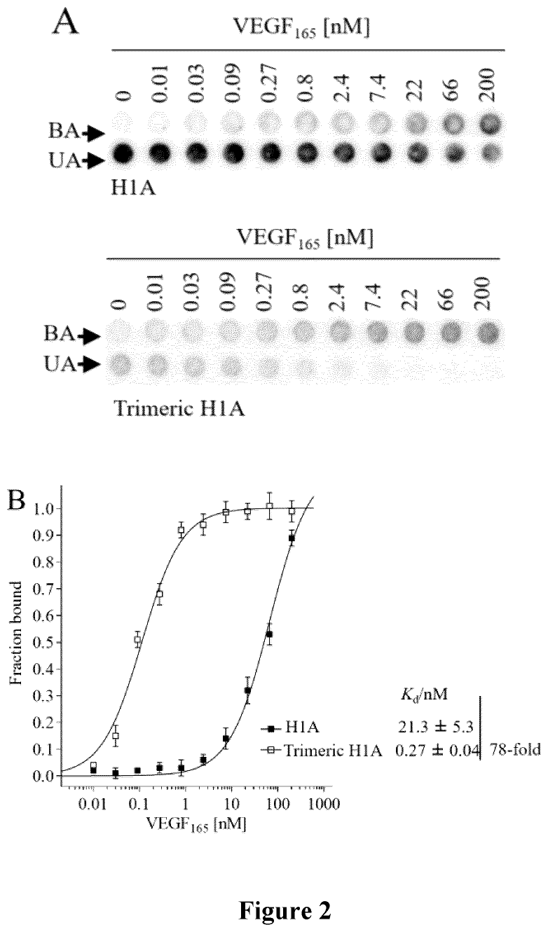

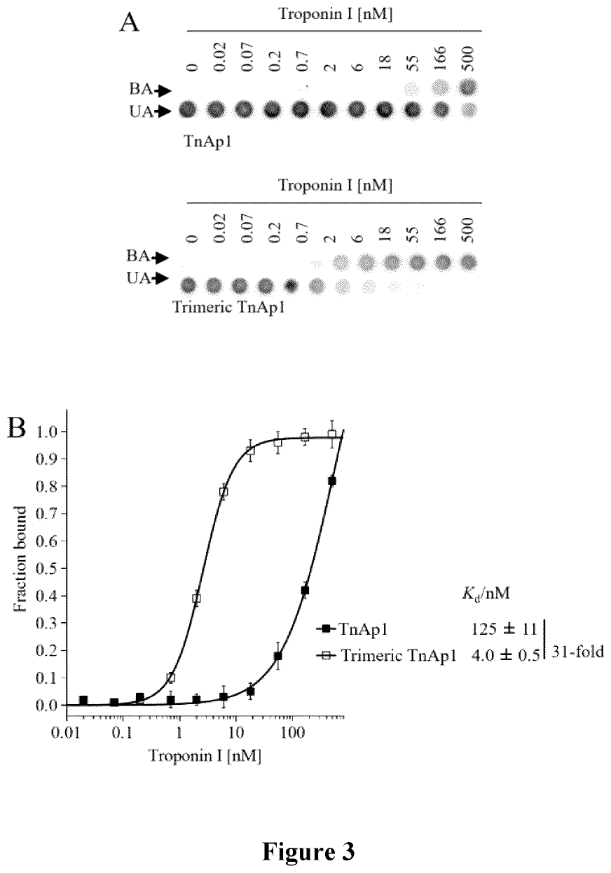

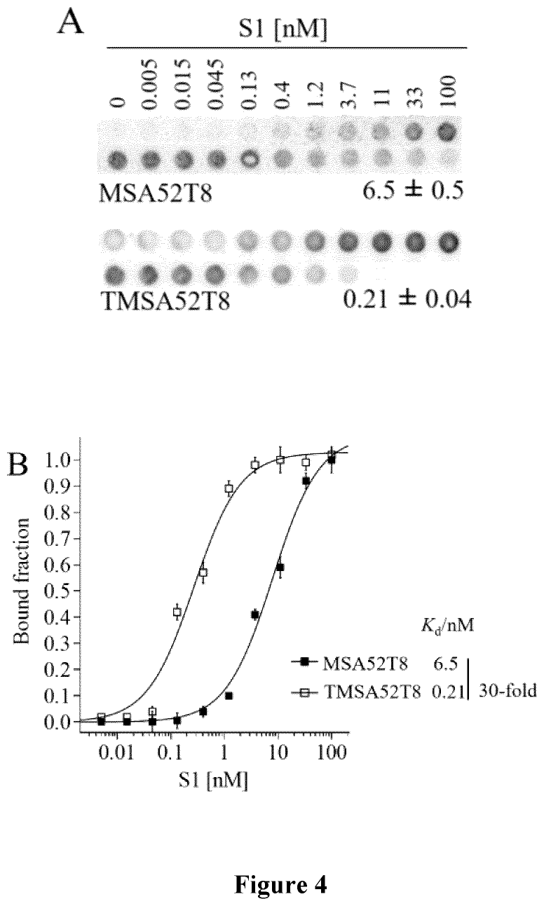

In addition to influenza HA, the trident aptamers for the three other biomarker proteins: VEGF, troponin-I, and SARS-COV-2 S1 subunit proteins was tested. Both the monomeric and trident aptamers for binding their respective target biomarker proteins were tested using the same dot blot assays, and the derived Kd values were used to evaluate the affinity enhancement provided by the trident aptamers. The results are shown in FIG. 2, FIG. 3, and FIG. 4, respectively.

By comparing the derived Kd values from the results, we observed significantly enhanced affinities for the three trident aptamers when binding to their target proteins. Specifically, the trident H1A aptamer for binding VEGF165 protein showed a Kd value of 0.27 nM, which is approximately 78-fold higher than the affinity improvement achieved by the monomeric H1A aptamer with a Kd value of 21.3 nM (FIG. 2). The trident TnAp1 aptamer for binding troponin-I protein demonstrated a Kd value of 4 nM, which is around 31-fold higher than its monomeric counterpart with a Kd value of 125 nM (FIG. 3). Finally, the trident aptamer TMSA52T8 for binding the S1 protein of SARS-COV-2 exhibited a Kd value of 0.21 nM, which is approximately 30-fold higher than the affinity achieved by the monomeric aptamer MSA52T8 with a Kd value of 6.5 nM (FIG. 4).

It is worth noting that while the influenza trident HA protein is a symmetric trimer, VEGF165 is a dimeric protein, and troponin-I and S1 of SARS-COV-2 are monomeric proteins. Despite this difference, enhanced affinities were still observed with these trident aptamers when binding to these proteins, indicating that the trident aptamers might be able to bind across their target proteins, and all three branches of the aptamers should remain functional.

Overall, four trident aptamers were tested for binding their respective biomarker proteins using dot blot assays. In comparison to their monomeric counterparts, these trident aptamers exhibited significant affinity enhancement, achieving improvements of 78-, 31-, and 30-fold, respectively. This trident aptamer approach could serve as a universal and powerful method for enhancing affinity in other aptamers as well.

These results confirm that the trident aptamer approach may be a universal method for significantly improving the binding affinity when recognizing protein biomarkers.

### Example 3: Cooperative Binding by Trident Aptamers

It was observed that trident aptamers exhibit high sensitivity for binding non-trimeric protein targets, even when unable to form the “three-to-three” symmetric structure between the aptamer and protein. We hypothesized that trident aptamers might have the ability to cross-bind target proteins, with all three branches of the trident aptamers cooperatively interacting with the proteins. To validate our hypothesis, we introduced the antisense (AS) DNA of each branch of the trident aptamers to block the binding between the branch and proteins. This blockade could result in an increase in the Kd value due to reduced affinity caused by the antisense DNA. We tested three trident aptamers—trident RHA06, trident H1A, and trident MSA52T8 (TMSA52T8)—that were synthesized using the above-described method, to bind their respective target proteins in different forms: trimeric (HA), dimeric (VEGF), and monomeric (S1). The samples were analyzed by native polyacrylamide gel electrophoresis (nPAGE).

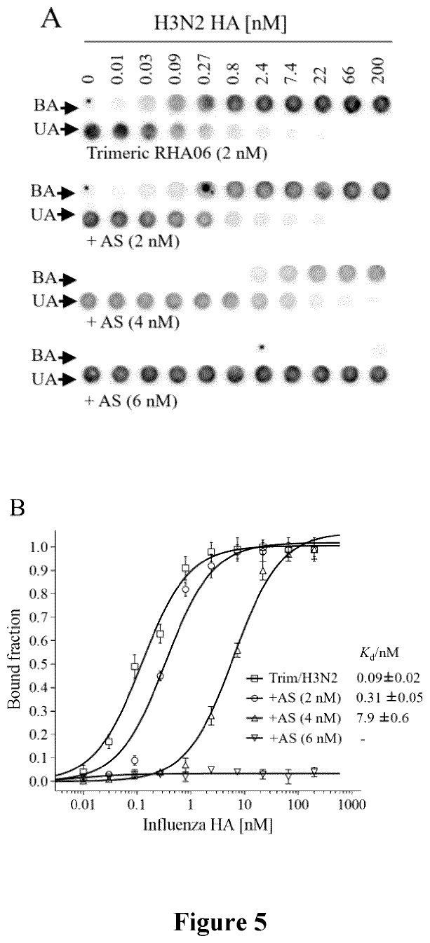

For the trident RHA06 aptamer binding to trimeric H3N2-HA proteins (FIG. 5), the binding activity of the aptamer gradually decreased as its antisense DNA was added, resulting in an increase in Kd values from 0.09 nM (AS:aptamer ratio=0:1) to 0.31 nM (AS:aptamer ratio=1:1) and 7.9 nM (AS:aptamer ratio=2:1). The Kd value at an AS:aptamer ratio of 2:1 was similar to the Kd value of the monomeric RHA06 binding to HA. When the AS:aptamer ratio reached 3:1, the binding affinity of the trident RHA06 aptamer was almost completely abolished. These results convincingly demonstrate that the three branches of the trident RHA06 aptamer cooperatively bind to the trimeric HA protein.

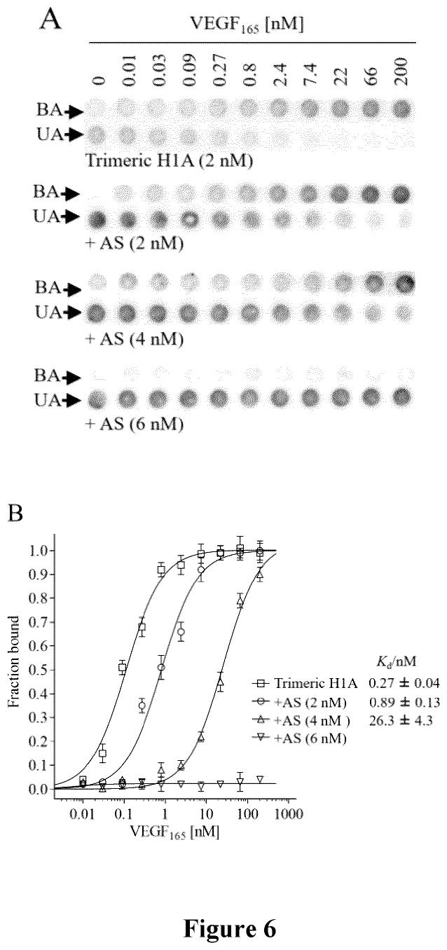

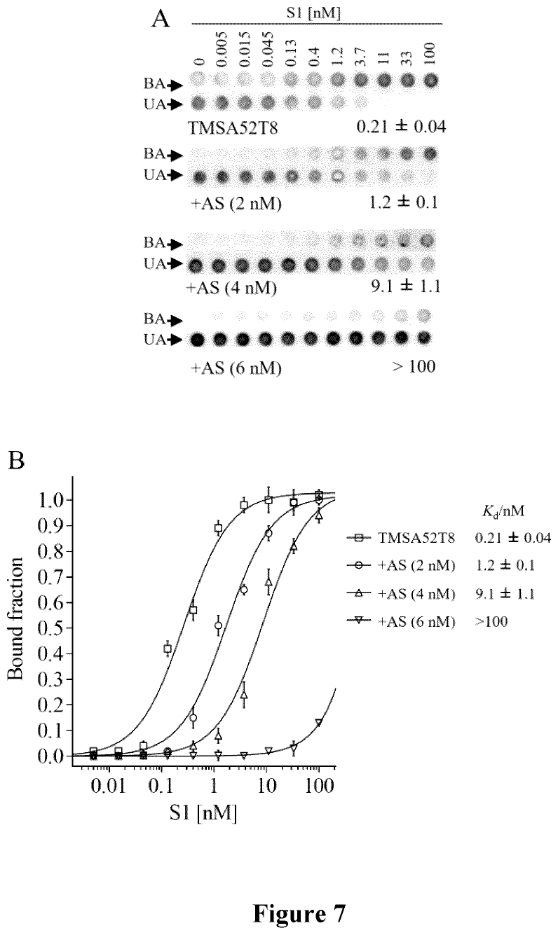

Similar results were also observed for trident aptamers binding to their non-trimeric protein targets. The affinity of the trident H1A aptamer gradually decreased with the addition of its antisense DNA, resulting in an increase in Kd values from 0.27 nM to 0.89 nM and 26.3 nM as the AS:aptamer ratio increased from 0:1 to 1:1 and 2:1 (FIG. 6). Affinity was completely lost when the ratio reached 3:1. Similar results were also observed for the trident aptamer TMSA52T8 binding to the monomeric S1 protein of SARS-COV-2 (FIG. 7), with the Kd value increasing from 0.21 nM to 1.2 nM, 9.1 nM, and >100 nM as the AS:aptamer ratio increased. These results confirm our hypothesis that the three branches of these trident aptamers can still functionally bind to non-trimeric protein targets without forming the symmetric three-to-three structures, suggesting that the aptamer branches cross-bind the proteins.

### Example 4: Kinetic Analysis of Trident Aptamer Binding

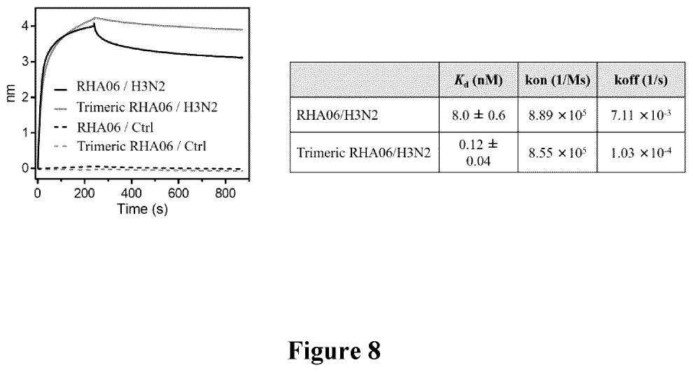

Following the confirmation of affinity enhancement in these trident aptamers, the factors contributing to the improved affinity was studied compared to the monomeric aptamers. The Bio-layer Interferometry (BLI) method was used to assess and compare the kinetic rates of the trident and monomeric aptamers. Since all four tested trident aptamers exhibited enhanced affinities compared to their monomeric counterparts, we focused our kinetic analysis on the aptamers binding to the influenza HA protein. The results and derived kinetic values are presented in FIG. 8.

Bio-layer Interferometry (BLI) tests. The binding of trident aptamers binding with protein was determined using the BLI Octet RED96. Octet® High Precision Streptavidin (SAX) Biosensors (Lot number: 2303010111, Sartorius AG) were first incubated in binding buffer for 10 min. Subsequently, the biosensors were loaded with biotin-labeled monomeric and trident aptamers by immersing them in 100 nM aptamer solutions. For the binding experiment, the H3N2-HA protein concentration was set at 200 nM. Basic Kinetics mode in the Octet Data Acquisition software was employed to define sample positions and assay steps, ensuring a significant decrease in signal during the dissociation phase, with a 15-minute duration for specific target affinity. Following the necessary procedures, including sensor ligation and reference signal subtraction, the signals were aligned to the baseline. Finally, a 1:1 binding model was fitted to the data. This streamlined approach effectively enabled the use of the Octet 96 instrument for protein binding assays involving biotin-labeled aptamers.

### Results

Both the monomeric and trident RHA06 aptamers displayed similar association rates (kₒₙ) of 8.89×10⁵ M⁻¹ s⁻¹ and 8.55×10⁵ M⁻¹ s⁻¹, respectively. This indicates that the monomeric aptamer was capable of binding to the protein as rapidly as the trident aptamer. However, the monomeric aptamer exhibited a significantly higher dissociation rate (k_{off}=7.11×10⁻³ s⁻¹), which was approximately 69-fold higher than that of the trident aptamer (k_{off}=1.03×10⁻⁴ s⁻¹). These results suggest that the presence of three branches in the trident aptamer allows for a much slower dissociation of the bound proteins compared to the monomeric aptamer, which only possesses a single binding branch. By calculating the K_{d} values from the BLI results using K_{d}=k_{off}/kₒₙ, we determined that the trident aptamer had a K_{d} value of 0.12 nM, which corresponds to an approximately 67-fold higher affinity than the monomeric aptamer with a K_{d} of 8.0 nM. These K_{d} values were consistent with those obtained from the dot blot assays (FIG. 1). In conclusion, the significantly enhanced affinity exhibited by the trident aptamers can be attributed to their reduced dissociation rates.

### Example 5: Preparation of Heterotrimeric Aptamer

A heterotrimeric aptamer was synthesized through a sequential ligation process, utilizing a 5′-phosphorylated trebler unit (TrU) as the scaffold. Each arm of the TrU was ligated with specific oligonucleotides: MSA1T, MSA5T, and MSA52T8, respectively. T4 DNA ligase was used for the ligation reactions, and templates LT1, LT2, and LT3 were used to guide the specific ligations for each arm (Table 2).

To phosphorylate TrU, 200 pmol of TrU was mixed with 10 U (U: enzyme unit) of T4 polynucleotide kinase (PNK) and 2 mM ATP in 50 μL of 1×PNK buffer A (50 mM Tris-HCl, pH 7.6, 10 mM MgCl₂, 5 mM DTT, 0.1 mM spermidine). The mixture was incubated at 37° C. for 1 hour, then heated at 90° C. for 5 min. For ligation of MSA1T to TrU, 200 pmol of MSA1T and 250 pmol of LT1 were added into the above reaction mixture, then heated at 90° C. for 5 min and cooled at room temperature for 20 min. To the above solution, 20 μL of 10×T4 DNA ligase buffer (400 mM Tris-HCl, 100 mM MgCl₂, 100 mM DTT, 5 mM ATP, pH 7.8) and 10 U of T4 DNA ligase were added. The resulting mixture, with a total volume of 200 μL, was incubated at room temperature for 2 hours before being heated to 90° C. for 5 minutes to deactivate the ligase enzyme. The mixture was subsequently concentrated using ethanol precipitation. The ligation product of MSA1T ligated with one arm of TrU (MSA1T-TrU) was then purified and isolated using 7.5% denaturing polyacrylamide gel electrophoresis (dPAGE). The prepared MSA1T-TrU was then ligated with MSA5T on the second arm of TrU using the template LT2, followed by the ligation of MSA52T8 onto the third arm using the template LT3. Finally, heterotrimers containing MSA1T, MSA5T, and MSA52T8 on each respective arm were purified and collected using 7.5% dPAGE. TrU as well as its single arm- and double arm-ligated products were employed as DNA markers during the dPAGE purification process.

TABLE 2
Heterotrimeric Aptamer Elements and Constructs

| | Size | Sequence (5′-3′) |
|---|---|---|
| Name | (nt) | italic nucleotides act as linkers with trebler |
| [symbol] | [symbol] | [symbol] |
| [symbol] | [symbol] | [symbol] |
| [symbol] | [symbol] | [symbol] |
| [symbol] | [symbol] | [symbol] |
| [symbol] | | |
| [symbol] | [symbol] | [symbol] |
| [symbol] | | [symbol] |
| [symbol] | | [symbol] |
| [symbol] | | [symbol] |
| [symbol] | [symbol] | [symbol] |
| [symbol] | [symbol] | [symbol] |
| [symbol] | [symbol] | [symbol] |

[symbol]

indicates data missing or illegible when filed

### Example 6: Assessment of Binding Affinity

Using dot blot assays, the binding affinity of the synthesized heterotrimeric aptamer, described in Example 5, for SARS-COV-2 S was assessed and compared to the binding affinities of the homotrimeric aptamer and monomeric aptamer.

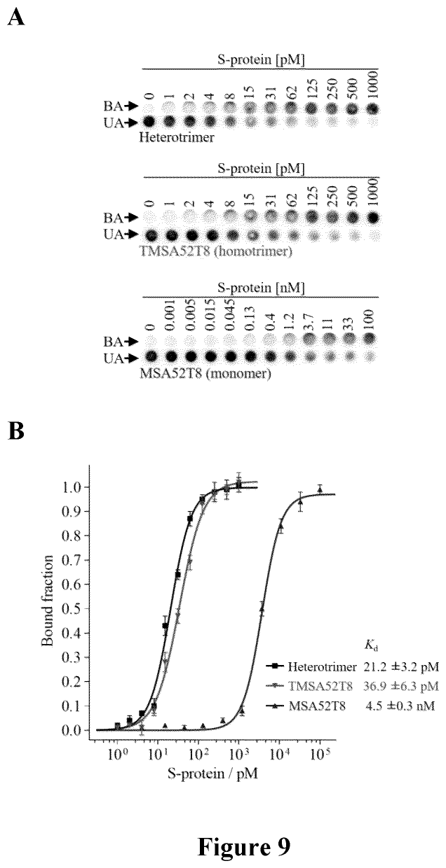

The results of the dot blots are provided in FIG. 9A, and the derived Kd values are shown in FIG. 9B. The heterotrimeric aptamer demonstrated comparable affinity for the S-protein of SARS-COV-2, as the homotrimeric aptamer, at the picomolar level. In particular, the heterotrimeric aptamer demonstrated significantly enhanced affinity, with a Kd value of 21.2 pM for the SARS-COV-2 S protein while the homotrimer aptamer demonstrated a Kd value of 36.9 pM. These affinities were 212- and 121-fold higher than those of the monomeric aptamer with a Kd value of 4.5 nM for the same protein. These results confirmed that the trident aptamer approach, whether hetrotrimeric or homotrimeric, can indeed enhance aptamer binding affinity.

### Example 7: Preparation of Homotrimeric Aptamer TMSA52

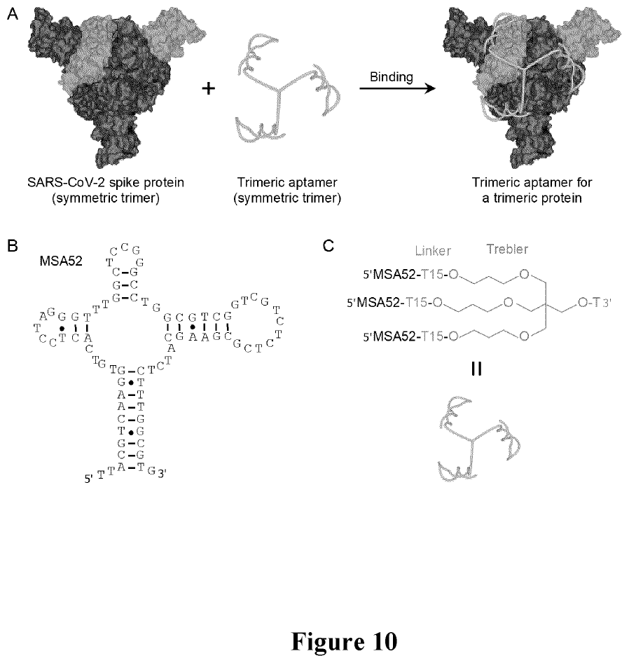

A homotrimeric aptamer for the trimeric spike protein of SARS-COV-2 was also prepared. The aptamer used was MSA52, a monomeric DNA aptamer MSA52 (FIG. 10B) discovered through selection with variant S proteins. MSA52 was found to universally recognize variants that were not analyzed in the original selection experiment, demonstrating that the aptamer is insensitive to emerging S protein mutations. Hence, MSA52 is an ideal candidate for COVID-19 recognition, and the trimerization of this MRE should only enhance its complementarity to the S protein.

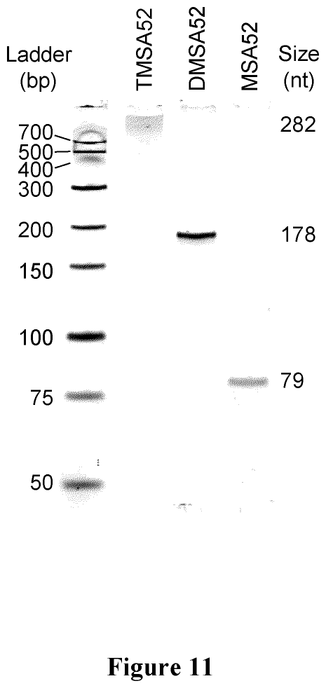

With the use of a 15-thymine linker and DNA synthesizer, the branched structural scaffold of a trebler was harnessed to synthesize a DNA molecule containing three identical MSA52 sequences (FIG. 10C), which is named TMSA52. Analysis with 10% denaturing polyacrylamide gel electrophoresis (dPAGE) of chemically synthesized TMSA52 showed that TMSA52 was synthesized successfully in reference to monomeric and dimeric MSA52 sequences (FIG. 11).

### Materials and Reagents

DNA oligonucleotides listed in Table 2 were obtained from Yale University or Integrated DNA Technologies and purified using 10% denaturing polyacrylamide gel electrophoresis (dPAGE) containing 8 M urea. Sodium borohydride (NaBH₄, 98%), sodium hexachloroiridate (III) hydrate (Na₃IrCl₆·xH₂O, M.W.=473.9), potassium phosphate monobasic (KH₂PO₄, ≥99%), sodium phosphate dibasic (Na₂HPO₄, ≥99%), potassium chloride (KCl, ≥99%), sodium chloride (NaCl, ≥99.5%), 4-(2-hydroxyethyl)-1-piperazineethanesulfonic acid (HEPES, ≥99%), magnesium chloride (MgCl₂, ≥99%), acetic acid (HOAc, ≥99.7%), 3,3′,5,5′-tetramethylbenzidine (TMB, >99%), sodium acetate (NaOAc, ≥99%), sulfuric acid (H₂SO₄, 95-98%), hydrogen peroxide solution (30% H₂O₂), dimethylformamide (DMF), streptavidin (Cat. No. SA101), bovine serum albumin (BSA, Cat. No. A7906), amylase (Cat. No. A1031), human IgG (Cat. No. 14506) and Tween-20 were all obtained from Sigma-Aldrich. The spike proteins of B.1.1.7 (Cat. No. SPN-C52H6), B.1.617.2 (Cat. No. SPN-C52He), B.1.617.1 (Cat. No. SPN-C52Hr), and B.1.1.529 (Cat. No. SPN-C52 Hz) SARS-COV-2 variants expressed in human 293 cells (HEK293) were obtained from Acro Biosystems. The spike proteins of B.1.351 (Cat. No. 510333-1), B.1.429 (Cat. No. 101057) and P.1 (Cat. No. 100989-1) SARS-COV-2 variants expressed in human 293 cells (HEK293) were obtained from BPS Biosciences Inc. The spike proteins for wild-type SARS-CoV-2 and SARS-COV-1, the spike protein RBD of seasonal coronavirus 229E and OC43, the control lentiviruses, the pseudotyped lentiviruses expressing the spike proteins of wild-type, B.1.351, and P.1 SARS-COV-2 were obtained from Dr. Matthew Miller's lab at McMaster University. The pseudotyped lentiviruses expressing the spike proteins of B.1.1.7 (Cat. No. 78112-1), B.1.617.1 (Cat. No. 78205-1), B.1.429 (Cat. No. 78172-1), B.1.617.2 (Cat. No. 78216-1), and B.1.1.529 (Cat. No. 78348-1) SARS-COV-2 were purchased from BPS Bioscience. Nitrocellulose membranes (Cat. No. 10600125) were from GE Healthcare Inc. Nylon membranes (Cat. No. NEF994001PK) were obtained from PerkinElmer Inc. The pooled human saliva (Lot 31887) was from Innovative Research Inc (Novi, Michigan). T4 polynucleotide kinase (PNK) with 10× buffer was acquired from Thermo Scientific (Ottawa, Canada). [γ-³²P]-ATP was purchased from PerkinElmer. 96-well microtiter plates (clear, polystyrene, flat bottom) were from Celltreat Inc. Ultrapure water (Milli-Q System, Millipore) was used to prepare all aqueous solutions.

TABLE 3
Synthetic DNA oligonucleotides. All sequences are written in a 5′ to 3′ direction.
Italic T segments act as linkers.

| SEQ | | Size | |
|---|---|---|---|
| NO IDs | Name | (nt) | Sequence |
| 1 | MSA52 | 79 | TTACGTCAAG GTGTCACTCC GTAGGGTTTG GCTCCGGGCC |
| | | | TGGCGTCGGT CGTCTCTCGC GAAGCATCTC TTTGGCGTG |
| 2 | MSA52-B | 84 | Biotin-TTTTT TTACGTCAAG GTGTCACTCC GTAGGGTTTG |
| | | | GCTCCGGGCC TGGCGTCGGT CGTCTCTCGC GAAGCATCTC |
| | | | TTTGGCGTG |
| 3 | DMSA52 | 178 | TTACGTCAAG GTGTCACTCC GTAGGGTTTG GCTCCGGGCC |
| | | | TGGCGTCGGT CGTCTCTCGC GAAGCATCTC TTTGGCGTG |
| | | | TTTTTTTTTT TTTTTTTTTT TTACGTCAAG GTGTCACTCC |
| | | | GTAGGGTTTG GCTCCGGGCC TGGCGTCGGT CGTCTCTCGC |
| | | | GAAGCATCTC TTTGGCGTG |
| 4 | DMSA52-B | 183 | Biotin-TTTTT TTACGTCAAG GTGTCACTCC GTAGGGTTTG |
| | | | GCTCCGGGCC TGGCGTCGGT CGTCTCTCGC GAAGCATCTC |
| | | | TTTGGCGTG TTTTTTTTTT TTTTTTTTTT TTACGTCAAG |
| | | | GTGTCACTCC GTAGGGTTTG GCTCCGGGCC TGGCGTCGGT |
| | | | CGTCTCTCGC GAAGCATCTC TTTGGCGTG |
| 5 | TMSA52 | 282 | TTACGTCAAG GTGTCACTCC GTAGGGTTTG GCTCCGGGCC |
| | | | TGGCGTCGGT CGTCTCTCGC |
| | | | GAAGCATCTC TTTGGCGTG TTTTTTTTTT TTTTT-Trebler |
| 6 | TMSA52-B | 287 | TTACGTCAAG GTGTCACTCC GTAGGGTTTG GCTCCGGGCC |
| | | | TGGCGTCGGT CGTCTCTCGC |
| | | | GAAGCATCTC TTTGGCGTG TTTTTTTTTT TTTTT-Trebler- |
| | | | TTTTT-Biotin |
| 7 | AS | 40 | GCGAGAGACG ACCGACGCCA GGCCCGGAGC CAAACCCTAC |

### Example 8: Assessment of Binding Affinity

Dot-blot binding assays of the trimeric aptamer. The binding affinities of the trimeric aptamer for spike proteins and pseudoviruses were tested by dot-blot binding assays. Briefly, nitrocellulose and nylon membranes were first immersed in binding buffer (50 mM HEPES, 150 mM NaCl, 6 mM KCl, 2.5 mM MgCl₂, 2.5 mM CaCl₂), and 0.01% Tween-20, pH 7.4) for 1 h. Then, the radioactive TMSA52 (10 μL, <0.1 nM) in the binding buffer was denatured at 90° C. for 5 min and annealed at 22° C. for 10 min. Different concentrations of spike proteins or pseudoviruses (10 μL) were mixed with the TMSA52 and incubated at 22° C. for 30 min. The reaction mixture was then filtered consecutively through a nitrocellulose membrane, a nylon membrane, and a wetted Whatman paper filter assembled on a Whatman Minifold-1 96-well apparatus using a vacuum pump. The bound aptamer on the nitrocellulose membrane and the unbound aptamer on the nylon membrane were developed on a storage phosphor screen for 12 h and observed using a Typhoon 9200 imager (GE Healthcare). The dot intensity was quantified with Image J software to determine the bound fraction of aptamer, which was plotted against the concentration of spike proteins or pseudoviruses. The K_{d} values were obtained via non-linear curve fitting using Origin 2020 software by the equation Y=BₘₐₓX/(K_{d}+X), where Y refers to the bound fraction of aptamer, Bₘₐₓ represents the maximum bound fraction of aptamer, and X stands for the concentration of spike proteins or pseudoviruses. The mixture of ³²P-labelled TMSA52 (10 pM) and unlabeled AS (different concentrations) was prepared to test the blocking effect of AS on the binding affinity of TMSA52.

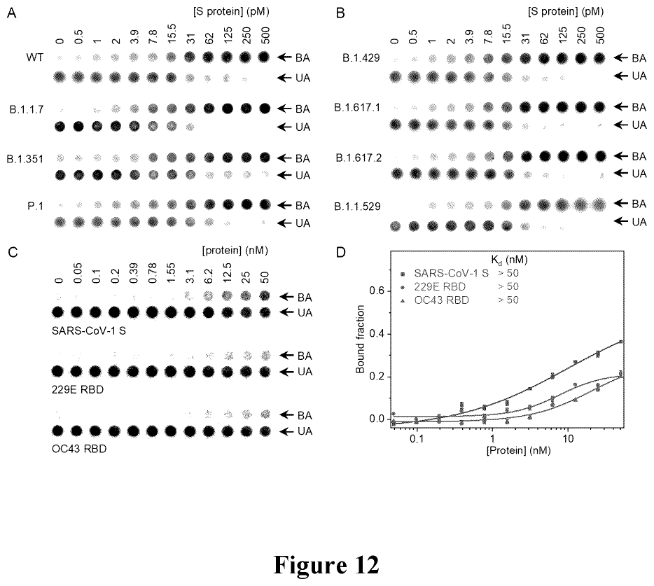

Assessment of binding affinity of TMSA52. Using a dot blot assay, the binding affinity of TMSA52 (sequence listed in Table 2) was first tested for eight different SARS-CoV-2 spike protein variants, including the WT (Wild-Type), B.1.1.7 (Alpha), B.1.351 (Beta), P.1 (Gamma), B.1.429 (Epsilon), B.1.617.1 (Kappa), B.1.617.2 (Delta), and B.1.1.529 (Omicron) variants were tested using dot-blot assays (FIG. 12, panels A and B). After labeling with ³²P at the 5′ end, TMSA52 was incubated with different concentrations of spike protein variants to form an aptamer/protein complex. The aptamer/protein complex was retained by a nitrocellulose membrane, whilst the free aptamer was collected on a nylon membrane. The concentration of bound aptamer on the nitrocellulose membrane and unbound aptamer on the nylon membrane were determined by their radioactivity.

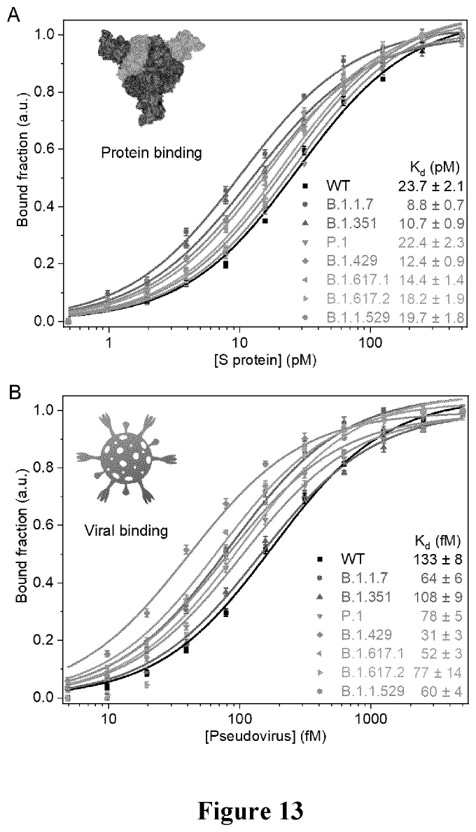

FIG. 13A plots the bound fraction of aptamer against the concentrations of each spike protein variant. The dissociation constants (K_{d} values) were obtained via non-linear curve fitting using the equation Y=BₘₐₓX/(K_{d}+X), where Y refers to the bound fraction of aptamer, Bₘₐₓ represents the maximum bound fraction of aptamer, and X stands for the concentration of spike proteins. The K_{d} values of TMSA52 for the eight spike protein variants ranged from 8.8 to 23.7 pM, which were approximately two orders of magnitude lower than for the corresponding monomeric aptamer MSA52. The significantly increased binding affinity was attributed to the superior trivalent interaction between the trimeric aptamer and the spike protein trimer.

TMSA52 was also tested for the binding of three control proteins including SARS-CoV-1 spike protein and spike RBD proteins of seasonal coronavirus 229E and OC43 (FIG. 12, panels C and D). The K_{d} values for the control proteins exceeded 50 nM, demonstrating the highly specific recognition ability of TMSA52 for the SARS-COV-2 spike proteins.

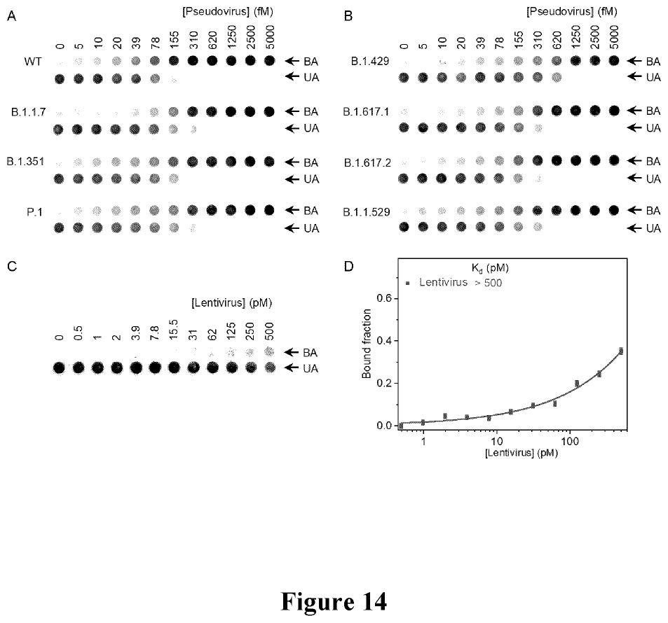

Following the same method, the binding affinities of TMSA52 were then tested for pseudotyped lentiviruses expressing the same spike protein variants, using a lentivirus without spike protein as a control (FIG. 14, panels A-C). The bound fraction of TMSA52 was plotted against the concentration of pseudoviruses to derive the K_{d} values (FIGS. 13B and 14D). The K_{d} values of TMSA52 for the eight pseudoviruses expressing different spike protein variants ranged from 31 to 133 fM, which was more than two orders of magnitude lower than for the monomeric aptamer MSA52. In contrast, the binding affinity for the control lentivirus was higher than 500 pM. Overall, the trimerization of MSA52 significantly increased the binding affinity for the recognition of all the SARS-COV-2 spike protein variants.

### Example 9: Cooperative Binding by Trident Aptamers

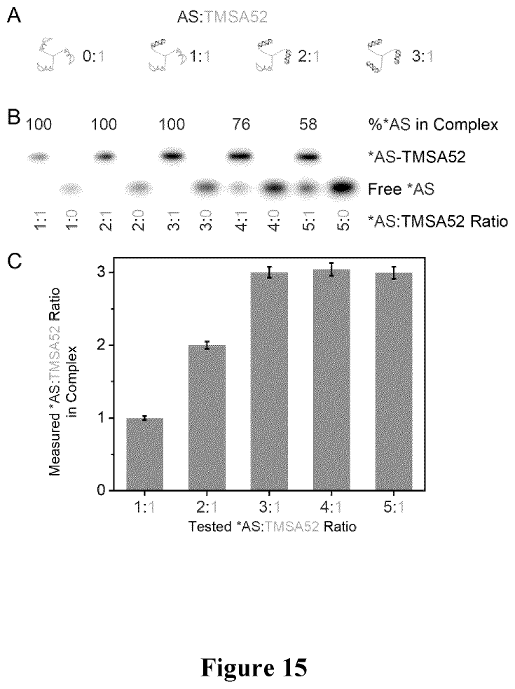

Cooperativity of binding by three aptamer arms. The next logical step was to validate the cooperative effect amongst the aptamer arms of TMSA52 for the binding of spike protein. By blocking the arms of TMSA52 with a 40-nt antisense (AS) DNA molecule (FIG. 15A), it can be assessed whether all three aptamer arms are required for the best possible binding. The precise AS:TMSA52 ratios were determined by an electrophoretic mobility shift assay, which used radioactive AS labeled with ³²P at the 5′ end. AS was mixed with TMSA52 in binding buffer at different ratios. After denaturation and annealing, the samples were analyzed by native polyacrylamide gel electrophoresis (nPAGE). As shown in FIG. 15 (panels B and C), ³²P-AS hybridized efficiently with TMSA52, but reached saturation at a ³²P-AS to TMSA52 ratio of 3:1. Further increases of ³²P-AS concentration resulted in no increase of binding number due to the limitation of three arms on each TMSA52.

### Materials and Reagents

Preparation of radioactive DNA. Trimeric aptamer TMSA52 was labeled with 32p at the 5′ end using PNK according to a previously reported protocol with slight modifications. 24 Briefly, TMSA52 (2 μL, 1 μM) was mixed with [γ-³²P]-ATP (1 μL), 10×PNK reaction buffer A (1 μL), PNK (1 μL, 10 U/mL) and water (5 μL) in a 200-μL PCR tube followed by incubation at 37° C. for 20 min. The reaction mixture was then purified by 10% dPAGE containing 8 M urea. Radioactive AS was prepared as described for TMSA52.

Electrophoretic mobility shift assay. The hybridization of TMSA52 with AS was tested by an electrophoretic mobility shift assay. Briefly, different concentrations of ³²P-labelled AS (10 μL) were mixed with unlabeled TMSA52 (10 μL, 10 pM) in the binding buffer. The mixture was denatured at 90° C. for 5 min and annealing at 22° C. for 10 min. After the addition of the loading buffer, the samples were analyzed using 10% native PAGE. The image of the gel was developed on a storage phosphor screen for 12 h and observed using a Typhoon 9200 imager.

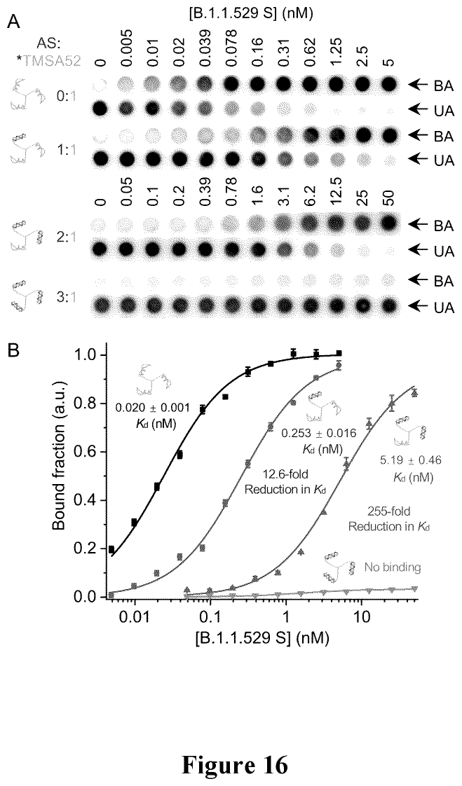

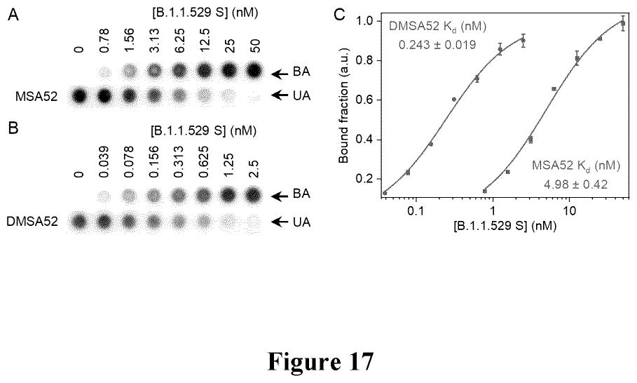

Assessment of binding affinity. Next the binding affinity of AS:TMSA52 complexes were investigated at different ratios for the spike protein. The B.1.1.529 variant spike protein was used for this experiment. The defined ratios of AS to ³²P-TMSA52 were mixed in binding buffer, followed by the addition of the B.1.1.529 spike protein. After a brief incubation at ambient temperature, the mixtures were analyzed by dot-blot assays. As shown in FIG. 16 panels A and B, the binding activity of TMSA52 was gradually reduced by AS with the K_{d} values increasing from 0.020 nM (AS:TMSA52=0:1) to 0.253 nM (AS:TMSA52=1:1) and 5.19 nM (AS:TMSA52=2:1). These values were consistent with the binding affinities of dimeric (DMSA52) and monomeric (MSA52) aptamers for the B.1.1.529 spike protein (FIG. 17). When the AS:TMSA52 ratio reached 3:1, the binding affinity of TMSA52 for the spike protein was almost completely abolished. These results adequately demonstrate that three TMSA52 arms associate with the three spike protein subunits in a concerted fashion. These results are consistent with the improved binding affinity of spherical aptamer, icosahedral DNA nanocage, and net-shaped DNA nanostructures for SARS-COV-2 spike protein.

### Example 10: Enzyme-Linked Aptamer Binding Assay (ELABA)

Design of an enzyme-linked aptamer binding assay. The universal recognition for spike variants by TMSA52 offers a solution to a significant COVID-19 complication—the continuing emergence of variants of concern. These variants have significantly hindered the sensitivity of current antigen-based rapid tests and increase the need for continuous adjustments with novel MREs. TMSA52 offers a promising breakthrough in COVID-19 detection, as it has been proven to recognize a vast array of SARS-COV-2 variants.

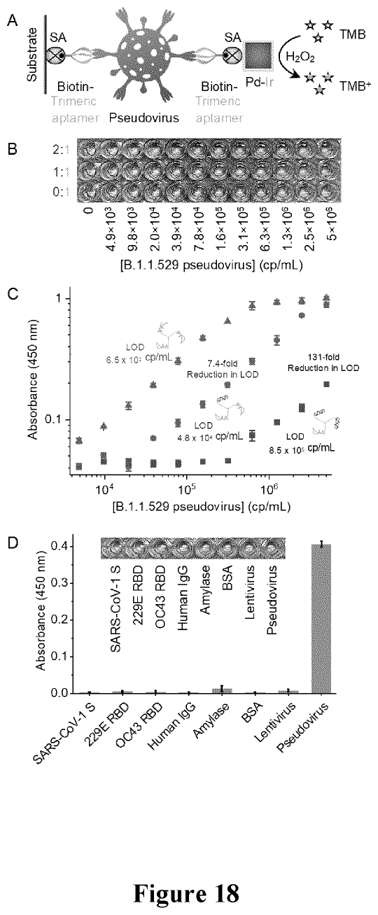

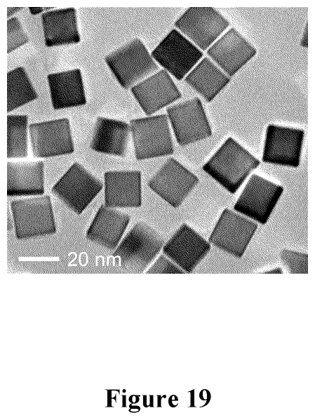

To employ the trimeric aptamer for the detection of all SARS-COV-2 variants in an easy, lab-ready format, a sandwich assay was utilized employing nanozymes, entities known for their high peroxidase-mimicking activity (FIG. 18A). Nanozymes are popular candidates to provide colorimetric signal outputs and greatly improve detection sensitivity. Thus far, the nanozymes with the highest peroxidase-mimicking activity are Pd—Ir nanocubes, which display approximately three orders of magnitude higher catalytic activity than horseradish peroxidase (HRP). To conduct an enzyme-linked aptamer binding assay (ELABA), a biotinylated trimeric aptamer (TMSA52-B, Table 2) was first attached to a streptavidin-coated microtiter plate or Pd—Ir nanocubes (synthesized according to previously reported methods; see FIG. 19.) through the biotin/streptavidin interaction, which is one of the strongest biomolecular interactions. SARS-COV-2 pseudovirus was then added to the aptamer-conjugated microtiter plate to bind with the immobilized trimeric aptamer. After washing, aptamer-conjugated Pd—Ir nanocubes were introduced to bind with the pseudovirus captured on the plate. The presence of SARS-COV-2 pseudovirus variants leads to the immobilization of aptamer-conjugated Pd—Ir nanocubes through a sandwich structure, which efficiently catalyzes the oxidation of colorless TMB with H₂O₂ to generate blue oxidized products. H₂SO₄ is then used to convert the TMB from blue to yellow. The concentration of the pseudovirus, which is proportional to the absorbance at 450 nm, can be easily determined by a plate reader.

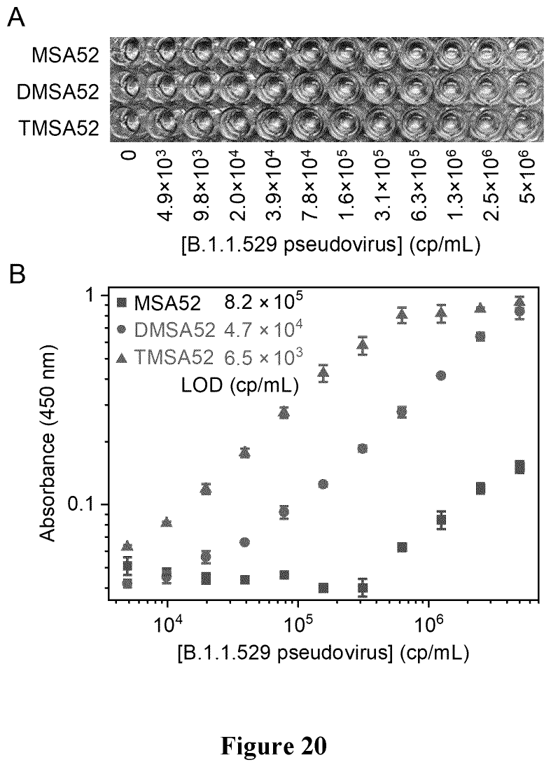

Using B.1.1.529 pseudovirus as a model target, the detection performance of trimeric (TMSA52-B), dimeric (DMSA52-B), and monomeric (MSA52-B) aptamer-based assays were compared. The ELABA procedures for dimeric and monomeric aptamers were the same as the above-described procedures for the trimeric aptamer, except for the substitution of TMSA52-B with DMSA52-B or MSA52-B. As shown in FIG. 20, the yellow (grey) intensity and absorbance at 450 nm increased proportionally with the concentration of the B.1.1.529 pseudovirus. The TMSA52-based method displayed a detection limit of 6.5×10³ cp/mL, which was 7.1-fold lower than DMSA52 (LOD 4.7×10⁴ cp/mL; LOD: limit of detection, 3-fold standard deviation of blank samples) and 126-fold lower than MSA52 (LOD 8.2×10⁵ cp/mL). The results demonstrate a significant improvement for the detection of pseudovirus by the trimerization of the original MSA52 aptamer.

To further verify the importance of trimeric binding for sensitive detection, AS was introduced to block the arms of TMSA52 prior to colorimetric detection. As shown in FIG. 18 panels B and C, the detection limit of the TMSA52-based assay increased from 6.5×10³ cp/mL to 4.8×10⁴ cp/mL and subsequently to 8.5×10⁵ cp/mL with the blocking of one and two TMSA52 arms, respectively. The results of increasing the AS:TMSA52 ratio were consistent with the DMA52 and MSA52-based assays (FIG. 20), demonstrating the importance of trimerization for enhancing the performance of biosensing assays.

Afterward, the specificity of the TMSA52-based assay for the detection of B.1.1.529 pseudoviruses was tested. SARS-COV-1 spike, spike RBD of seasonal coronaviruses 229E and OC43, human IgG, amylase, BSA, and lentivirus were used as controls. The concentrations of control protein (10 nM) were approximately 5 orders of magnitude higher than B.1.1.529 pseudoviruses (120 fM). As shown in FIG. 18D, the assay was capable of specifically detecting B.1.1.529 pseudoviruses with a negligible signal response for the control proteins or lentivirus. These results strongly support the practicality of TMSA52 as a COVID-19 MRE as demonstrated by its exceptional binding affinity and specificity.

### Example 11: Detection of Pseudoviruses Spiked in Human Saliva

ELABA for the detection of psuedoviruses using trimeric aptamer and Pd—Ir nanocubes. Pseudoviruses were detected using the trimeric aptamer and Pd—Ir nanocube-based enzyme-linked aptamer binding assay (ELABA). Pd—Ir nanocubes with an edge length of 18 nm were prepared according to previously reported methods. The concentration of Pd—Ir nanocubes was 2.2 nM. Pd—Ir nanocubes were then conjugated with streptavidin through physical adsorption. Briefly, streptavidin (10 μL, 1 mg/mL) and mPEG-SH (10 μl, 100 μM) were added to the Pd—Ir nanocube solution (1 mL, 0.55 nM) and incubated at 22° C. for 1 h. After centrifuging at 10000 rpm for 5 min and washing once with PBST (PBS: 137 mM NaCl, 2.7 mM KCl, 8 mM Na₂HPO₄, and 2 mM KH₂PO₄, pH 7.4, containing 0.05% Tween-20), TMSA52-B (100 μL, 2 μM) in PBST was added and incubated at 22° C. for 30 min. Afterward, BSA (200 μL, 10% w/v) solution was added to the mixture and incubated at 22° C. for 1 h to block the Pd—Ir nanocubes. Aptamer-conjugated Pd—Ir nanocubes were pelleted by centrifuging at 10000 rpm for 5 min and washed twice with BSA solution (1 mL, 1% w/v). Thereafter, aptamer-conjugated Pd—Ir nanocubes were resuspended in PBST containing 10% w/v BSA, followed by storage at 4° C. for 12 h before use.

Subsequently, a 96-well microtiter plate was coated with streptavidin (100 μL, 5 μg/mL) in PBS by incubation at 4° C. for 12 h, followed by TMSA52-B conjugation (100 μL, 200 nM) in PBS at 22° C. for 30 min, and then blocking with BSA (100 μL, 2% w/v) in PBS at 37° C. for 1 h. After one wash with PBST (300 μL), different concentrations of pseudoviruses (100 μL) spiked in 25% saliva in PBST were added to the wells of aptamer-coated 96-well plate and incubated at 22° C. for 30 min. The plate wells were washed once with PBST (300 μL). Aptamer-conjugated Pd—Ir nanocubes (100 μL, 0.138 nM) in PBST were then added and incubated at 22° C. for 30 min. The wells were then washed four times with PBST (300 μL). Finally, TMB substrate solution (100 μL, 0.8 mM TMB, 2 M H₂O₂, 0.1 M HACO/NaAcO buffer, pH 4) was added and reacted at 22° C. for 10 min, followed by terminating the catalytic reaction with H₂SO₄ (20 μL, 2 M). The absorbance of the oxidized TMB product at 450 nm was measured using a plate reader (Tecan, Switzerland). The protocols of MSA52 or DSA52-based detection were the same as above-described with the substitution of biotinylated trimeric aptamer (TMSA52-B) with monomeric (MSA52-B) or dimeric (DSA52-B) aptamers. To conduct the AS and TMSA52 mixture-based detection, different concentrations of AS (50 μL) were mixed with TMSA52-B (50 μL, 400 nM) in PBST buffer, followed by denaturing at 90° C. for 3 min and annealing at 22° C. for 10 min. Then, the mixture was used to substitute the biotinylated trimeric aptamer for the detection of pseudovirus as described above. To detect SARS-COV-2 in clinical saliva samples by ELABA, saliva was first diluted by PBST to 25%, then added to the aptamer-coated plates and detected as described above.

ELABA for the detection of SARS-COV-2 in patient saliva samples. Collection of saliva specimens was performed using a protocol approved by the Hamilton Integrated Research Ethics Board (HiREB Project #12636). Patients attending COVID-19 assessment centers at sites operated by Hamilton Health Sciences or St. Joseph's Healthcare in Hamilton, Ontario were invited to donate a supervised, self-collected, drool saliva sample immediately following collection of a nasopharyngeal swab (NPS). Saliva specimens were stored at 4° C. during transport (<72 hours) and subsequently long-term at −80° C. NPS specimens collected for standard COVID-19 screening were tested using a standard RT-PCR method by the Hamilton Regional Laboratory Medicine Program at St. Joseph's Healthcare Hamilton. NPS test results were used to identity candidate negative (60 patients, NS1-60), positive saliva specimens (27 patients, PS1-27) and to assign putative variants to each positive sample. The presence of SARS-COV-2 in candidate saliva specimens was further confirmed using a saliva RT-PCR method described previously.

All patient saliva samples were diluted to 25% (v/v) using PBST and were tested using the ELABA method described above. Control reactions included a positive control (PC) containing 4×10⁴ cp/mL of B.1.1.529 pseudovirus spiked in commercial pooled human saliva and diluted to 25% (v/v) with PBST and a negative control (NC) consisting of commercial pooled human saliva diluted to 25% (v/v) with PBST. Data was plotted using a Receiver-Operator Characteristic plot and a cut-off was determined as the value that maximized the sum of Sensitivity and Specificity to determine the number of true positives and true negatives identified with the assay.

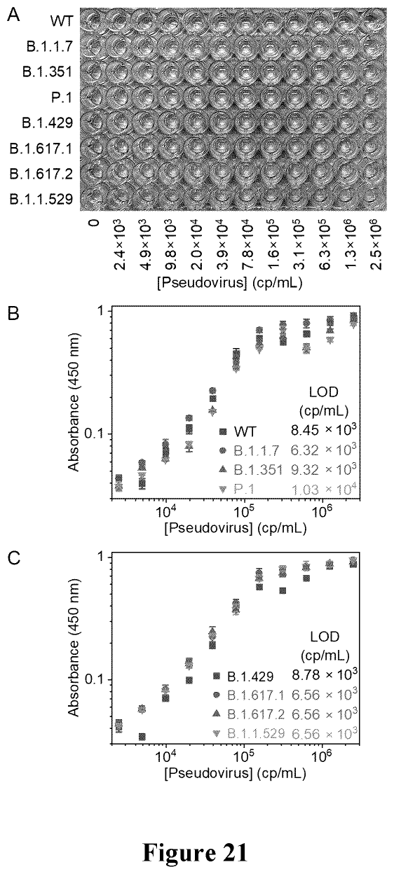

Detection of pseudoviruses spiked in human saliva. Next, the TMSA52-based biosensor was employed for the detection of eight SARS-COV-2 spike variant pseudoviruses spiked in 25% pooled human saliva. Once again, the yellow (grey) intensity and absorbance at 450 nm increased proportionally with the pseudovirus concentration (FIG. 21). The limit of detection (LOD,) ranged between 6.3×10³ to 1.0×10⁴ cp/mL for all eight pseudovirus variants, with the highest detection sensitivity for the B. 1.1.7 variant and the lowest detection sensitivity for the P.1 variant. These results highlight the universal recognition capabilities of the TMSA52 aptamer.

### Example 12: Evaluation of the Biosensor Using Clinical Samples

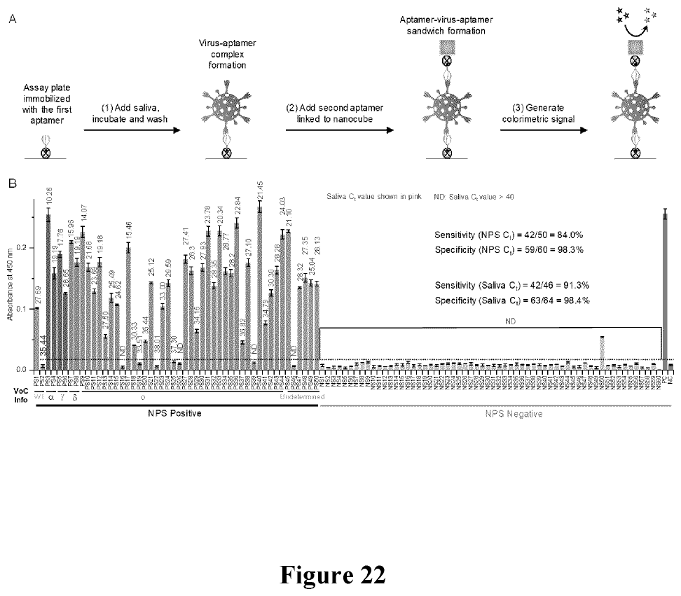

Evaluation of the biosensor using clinical samples. To evaluate the clinical utility of the sandwich assay employing the trimeric aptamer, a panel of 110 patient saliva samples were examined, including 50 NPS positive and 60 NPS negative samples. Table 3 provides details on each sample, including NPS and saliva-based Ct values obtained from RT-PCR along with the presumed variant (for positive samples). FIG. 22A shows the assay workflow. TMSA52-B was first immobilized to the surface of the microwell. Saliva samples were diluted to 25% using assay buffer and then incubated with the TMSA52-B aptamer (30 min) followed by a washing step to capture the virus on the microwell surface. The detection TMSA52-B aptamer, pre-bound to Pd—Ir nanoplates, was then added and allowed to incubate for 30 min prior to washing. TMB was then added, and the signal was measured after 10 min. The total sample to readout time was 70 min. However, a total of 96 samples can be analyzed simultaneously by this method, producing an assay time of under one minute per sample.

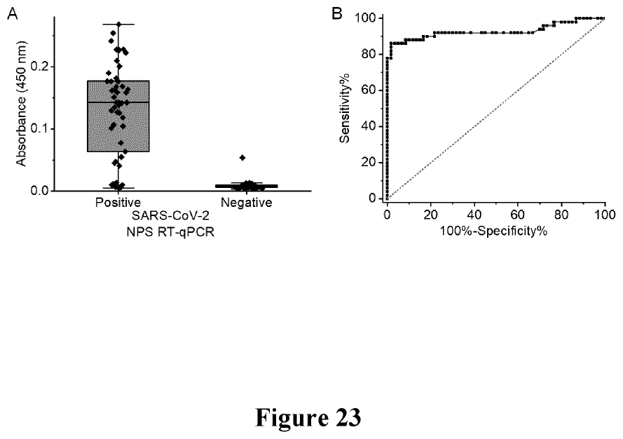

FIG. 22B shows the absorbance values measured for each of the individual positive (PS #) and negative (NS #) saliva samples, along with high (PC) and low (NC) controls. The Ct value for each positive saliva sample is shown above its respective bar. Based on a Receiver Operating Characteristic (ROC) curve (FIG. 23), the clinical cut-off absorbance (A450) value was determined to be 0.027, which resulted in a sensitivity of 84.0% true positives detected, a specificity of 98.3% true negatives detected, and an overall accuracy of 92.9%. It is noted that while each of the eight misidentified samples was identified as positive using RT-PCR of NPS samples (though all with Ct values over 33), four of these samples did not show detectable RNA in the corresponding saliva sample, hence the clinical sensitivity based on a comparison to saliva RT-PCR data rises to 91.3% if these four samples are considered as negatives.

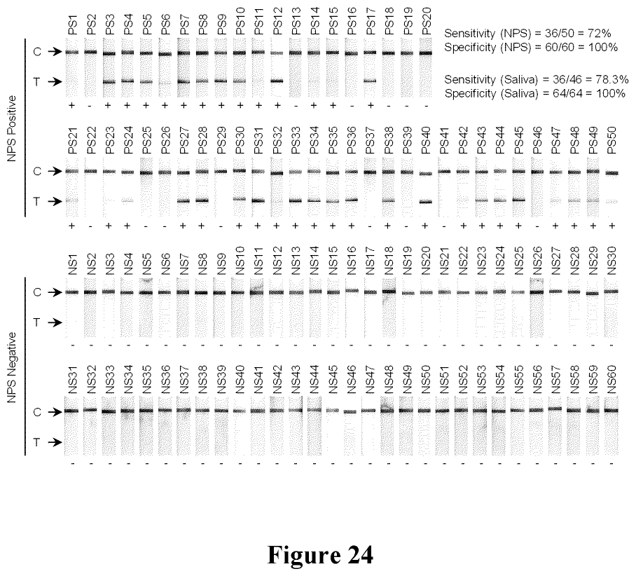

An important point from FIG. 22B is that the detection of SARS-COV-2 in positive patient saliva does not depend on the variant. This clearly shows the key advantage of using the TMSA52 aptamer as it can produce positive signals regardless of the variant. The data also show that negative patient saliva samples do not contribute to significant background signals. This further supports the high selectivity of the TMSA52 aptamer, as it is insensitive to potential interferants that might be present in-patient saliva. As a comparison, the 110 patient saliva samples were also tested by BTNX COVID-19 antigen rapid test. As shown in FIG. 24, the rapid test showed a detection sensitivity (NPS) of 72%, which was lower than the TMSA52-based ELABA method.

BTNX COVID-19 antigen rapid test for saliva samples. The saliva sample (30 μL) was mixed with detection buffer (270 μL), followed by addition to the well of BTNX COVID-19 antigen rapid test device (REF: COV-19C25). After reaction for 20 min, the photograph of the test result was captured with a cellphone and processed with Photoshop 2020.

### Conclusion

To engineer the novel MRE, a trebler and linker system were adopted, whilst utilizing the pre-existing, universal aptamer MSA52. TMSA52 can recognize the most notable spike protein variants, including the wild-type, B.1.1.7 (Alpha), B.1.351 (Beta), P.1 (Gamma), B.1.429 (Epsilon), B.1.617.1 (Kappa), B.1.617.2 (Delta), and recent B.1.1.529 (Omicron) variants, with K_{d} values ranging from 8.8 to 23.7 pM. Compared to its monomeric and dimeric equivalents, TMSA52 exhibited increased binding affinity towards both the S protein and pseudovirus samples. The exceptional recognition was attributed to the symmetrical, multivalent interaction between the trimeric aptamer and spike protein trimer. For the application of the aptamer into a practical setting, a colorimetric assay for the detection of SARS-COV-2 variants was developed using Pd—Ir nanocubes as peroxidase mimicking nanozymes for signal output. Eight pseudoviruses displaying different SARS-COV-2 spike variants in pooled human saliva have been specifically identified with detection limits ranging from 6.3×10³ to 1.0×10⁴ cp/mL. Finally, the assay was applied to the detection of SARS-CoV-2 in patient saliva samples, providing a clinical sensitivity of 84.0% and specificity of 98.3% compared to RT-PCR of NPS samples.

The trimeric aptamer shape is a perfect fit for the trimeric S protein, granting it an affinity and specificity that is unique relative to any other aptamer published in the literature. TMSA52 holds great potential for broader diagnostic and therapeutic COVID-19 applications and, moving forward, should serve as the dominant choice of all SARS-COV-2 MREs. The precision engineering of a trimeric aptamer specifically tailored to a trimeric protein provides a new approach for engineering high performing MREs for a wide range of target molecules.

TABLE 4
Comparative study of the positive COVID-19 saliva samples using
trimeric aptamer-based assay (ELABA), BTNX COVID-19 antigen
rapid test, NPS Ct (RT-PCR), and saliva Ct (RT-PCR).

| Sample | | | NPS | Saliva | |
|---|---|---|---|---|---|
| ID | ELABA | Panbio | Ct | Ct | Variant |
| PS1 | + | + | 19.23 | 27.69 | WT |
| PS2 | − | − | 33.16 | 35.44 | WT |
| PS3 | + | + | 22.26 | 10.26 | B.1.1.7 |
| PS4 | + | + | 21.32 | 19.19 | B.1.1.7 |
| PS5 | + | + | 13.37 | 17.76 | P.1 |
| PS6 | + | + | 13.48 | 28.65 | P.1 |
| PS7 | + | + | 14.19 | 15.96 | B.1.617.2 |
| PS8 | + | + | 19.44 | 19.19 | B.1.617.2 |
| PS9 | + | + | 16.87 | 14.07 | B.1.1.529 |
| PS10 | + | + | 1.18 | 21.68 | B.1.1.529 |
| PS11 | + | + | 14.27 | 23.69 | B.1.1.529 |
| PS12 | + | + | 21.40 | 19.18 | B.1.1.529 |
| PS13 | + | − | 17.33 | 27.50 | B.1.1.529 |
| PS14 | + | + | 17.38 | 25.49 | B.1.1.529 |
| PS15 | + | + | 27.40 | 24.62 | B.1.1.529 |
| PS16 | − | − | 29.60 | ND | B.1.1.529 |
| PS17 | + | + | 16.78 | 15.46 | B.1.1.529 |
| PS18 | + | − | 15.13 | 30.33 | B.1.1.529 |
| PS19 | − | − | 27.28 | 33.51 | B.1.1.529 |
| PS20 | + | − | 18.75 | 36.44 | B.1.1.529 |
| PS21 | + | + | 13.11 | 25.12 | B.1.1.529 |
| PS22 | − | − | 14.13 | 38.01 | BA.2 |
| PS23 | + | + | 32.48 | 33.00 | BA.2 |
| PS24 | + | + | 16.56 | 29.59 | BA.4 |
| PS25 | − | − | 16.30 | 37.30 | BA.4 |
| PS26 | − | − | 23.08 | ND | BA.4 |
| PS27 | + | + | 20.97 | 27.41 | BA.5 |
| PS28 | + | + | 18.22 | 26.30 | BA.5 |
| PS29 | + | − | 15.25 | 34.16 | BA.5 |
| PS30 | + | + | 15.68 | 27.93 | BA.5 |
| PS31 | + | + | 17.06 | 23.78 | BA.5 |
| PS32 | + | + | 22.26 | 28.35 | BA.5 |
| PS33 | + | + | 15.76 | 20.34 | BA.5 |
| PS34 | + | + | 15.08 | 28.77 | BA.5 |
| PS35 | + | + | 20.42 | 28.20 | BA.5 |
| PS36 | + | + | 18.59 | 22.84 | BA.5 |
| PS37 | + | − | 14.67 | 36.82 | BA.2.12.1 |
| PS38 | + | + | 19.49 | 27.10 | BA.2.12.1 |
| PS39 | − | − | 18.10 | ND | BA.2.12.1 |
| PS40 | + | + | 23.56 | 21.45 | BA.2.12.1 |
| PS41 | + | − | 12.38 | 34.79 | BA.2.12.1 |
| PS42 | + | + | 15.71 | 30.30 | BA.2.12.1 |
| PS43 | + | + | 18.21 | 28.28 | BA.2.12.1 |
| PS44 | + | + | 24.53 | 24.03 | BA.2.12.1 |
| PS45 | + | + | 26.14 | 21.10 | Undetermined |
| PS46 | − | − | 31.74 | ND | Undetermined |
| PS47 | + | + | 24.95 | 28.32 | Undetermined |
| PS48 | + | + | 16.50 | 27.35 | Undetermined |
| PS49 | + | + | 15.39 | 25.04 | Undetermined |
| PS50 | + | + | 14.40 | 28.13 | Undetermined |

Note:
‘+’ standards for positive results, whereas ‘−’ refers to negative results by trimeric aptamer-based assay.
PS: Positive saliva samples.

### Example 13: In Vitro Assessment of Aptamers in Neutralizing SARS-CoV-2

### In Vitro Neutralization Assay

Vero E6 cells (ATCC CRL-1586) were seeded at a density of 1.5×10⁴ cells/well in white flat-bottom TC-treated 96-well plates (Corning, 3917) and incubated at 37° C., 5% CO₂. On the day of seeding, at the end of the day, the medium was replenished with fresh DMEM fortified with 2% FBS, 1% Penicillin-Streptomycin, 1% HEPES (pH=7.3), and 1% Glutamax. The cells were then further incubated for 24 hours. Meanwhile, aptamers (TMSA52, Ir-TMSA52) and nanoplate (Ir only) were serially diluted down each column in an empty 96-well plate, starting with a 1:4 (BA.1) and 1:2 (MA10) dilution from a 1 μM stock solution and monoclonal antibody (S309) starting with 1 μg/ml. These aptamer dilutions were incubated with the ancestral strain and Omicron Variant (Lineage BA.1) of SARS-COV-2 (330 plaque-forming units (PFU)/well) for 1 hour at 37° C., 5% CO₂. Following this incubation, the mixture was transferred onto the Vero E6 cells and re-incubated for 1 hour under the same conditions. The mixture was then replaced with identical dilutions of the aptamers and incubated for a further 72 hours at 37° C., 5% CO₂. Post-incubation, cell viability was ascertained using the CellTiter-Glo 2.0 Luminescent Cell Viability Assay Kit (Promega), where luminescence intensity was directly proportional to the number of viable cells. Luminescence was quantified using a BioTek Synergy H1 microplate reader, and neutralization titer was determined as the highest aptamer/antibody dilution that achieved a 50% reduction in luminescence compared to the virus control wells.

### Results

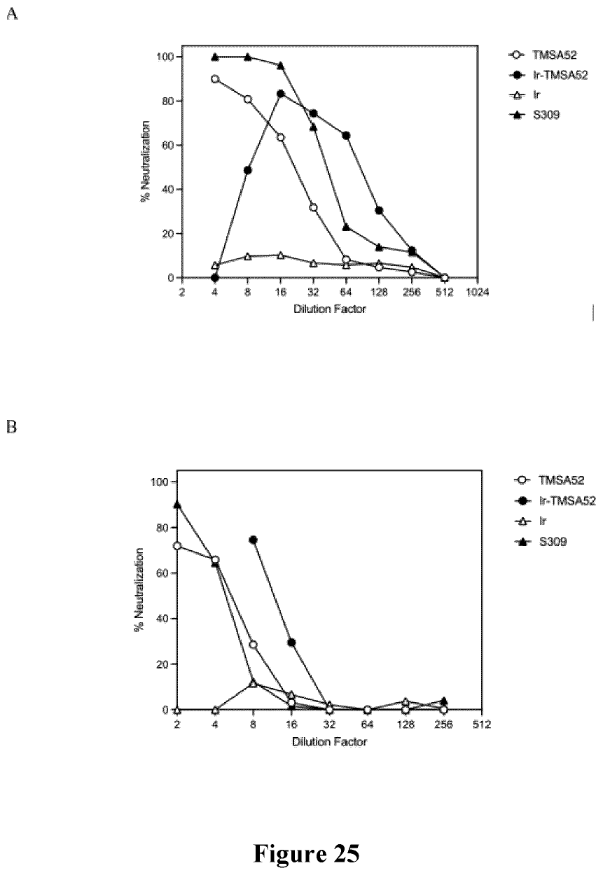

By utilizing a well-established microneutralization (MNT) assay, we assessed our aptamers in neutralizing SARS-COV-2. Given the universality of our trimeric aptamer (TMSA52) or iridium nanoplate-scaffolded aptamer (Ir-TMSA52) in binding to the spike protein, we first quantified aptamer neutralization utilizing the immune evasive Omicron (BA.1) variant of SARS-COV-2 and compared it to a clinically approved SARS-COV-2 monoclonal antibody, S309 (FIG. 25A). In accordance with published data, S309 was capable of robustly neutralizing SARS-COV-2 Omicron BA.1 (FIG. 25A, closed triangles). Impressively, both TMSA52 (FIG. 25A, open circles) and the iridium nanoplate-scaffolded Ir-TMSA52 (FIG. 25A, closed circles) both showed comparable neutralization activity to S309. The iridium nanoplate alone failed to confer any neutralizing activity (FIG. 25, open triangles). Given the robust neutralizing ability of our aptamers, we next assessed neutralization against a mouse-adapted variant of SARS-COV-2 (MA10), preceding our downstream in vivo studies with the same virus strain. In accordance with the neutralization observed with SARS-COV-2 Omicron BA.1, S309 was capable of robustly neutralizing SARS-COV-2 MA10 (FIG. 25B, closed triangles). Impressively, both TMSA52 (FIG. 25B, open circles) and the iridium nanoplate-scaffolded Ir-TMSA52 (FIG. 25B, closed circles) both showed comparable neutralizing activity as S309. The iridium nanoplate alone failed to confer any neutralizing activity (FIG. 25B, open triangles).

### Conclusions

Our aptamers, both the trimeric (TMSA52) and iridium nanoplate-scaffolded (Ir-TMSA52) demonstrate significant neutralization capabilities against SARS-COV-2, matching the performance of the clinically approved monoclonal antibody S309. These results highlight the robust potential of our aptamers as effective countermeasures against SARS-COV-2 variants, showcasing their comparability to established therapeutic standards.

### Example 11: In Vivo Assessment of Aptamers in Protection Against Non-Lethal Challenge with SARS-CoV-2

### Treatment Types and Delivery

Aptamers, nanoplates, and antibodies were synthesized in vitro and diluted to desired concentrations in nuclease-free water. A protective dose of intranasally (i.n.) administered S309 monoclonal antibody was previously determined (data not shown), and equimolar concentrations of aptamer were calculated for synthesis. Mice were deeply anesthetized by isoflurane inhalation and administered the desired treatment in a final volume of 40 μL (20 μL bolus to each nostril) two hours prior to infection.

### Virus Strains and Delivery

Wild-type SARS-COV-2 (USA-WA1/2020) was serial passaged 10 times in BALB/c mice to generate SARS-COV-2 (MA10), which was generously provided by Dr. Ralph Baric. Age-matched 6-8-week-old wild-type female BALB/c mice were purchased from Charles River Laboratories (Saint Constant, QC, Canada). Mice were deeply anesthetized by isoflurane inhalation, and infected i.n. with SARS-COV-2 in a final volume of 40 μL (20 μL bolus to each nostril). Animals were housed in either a specific pathogen-free level B or a Containment Level 3 Facility at McMaster University, Hamilton, ON, Canada. All experiments were performed in accordance with institutional guidelines from the Animal Research and Ethics Board.

### Weight Loss and Clinical Scoring

Prior to treatment, mice were weighed to determine their initial weight as a benchmark. Mice were monitored for clinical signs and weight loss daily, with 80% of initial weight considered humane endpoint in accordance with institutional guidelines. Mice were scored for the presence of clinical signs, with one point being administered for ruffled fur, rapid breathing, hunched back, lethargy.

### SARS-COV-2 Viral Burden Determination in Tissues

Vero E6 (CRL-1586, American Type Culture Collection (ATCC), Manassas, VA, United States) were cultured at 37° C. in Dulbecco's Modified Eagle medium (DMEM) supplemented with 10% fetal bovine serum (FBS), 1% HEPES (pH=7.3), 1 mM sodium pyruvate, 1% L-Glutamine and 100 U/mL of penicillin-streptomycin. Lungs were homogenized using a Bead Mill 24 homogenizer (ThermoFisher Scientific Waltham, MA, United States) and frozen at −80° C. Homogenates were thawed and clarified by centrifugation at 300×g. Homogenates were serially diluted 1:10 in low-serum DMEM supplemented with 2% FBS, 1% HEPES (pH=7.3), 1 mM sodium pyruvate, 1% L-Glutamine and 100 U/mL of penicillin-streptomycin. 100 μL of viral inoculum was transferred onto Vero E6 cells seeded the day before in 96-well plates (2.5×10⁴ cells per well). Wells were visually assessed for cytopathic effect at 72 hours post-infection using an EVOS M5000 microscope (ThermoFisher Scientific Waltham, MA, United States).

### Results

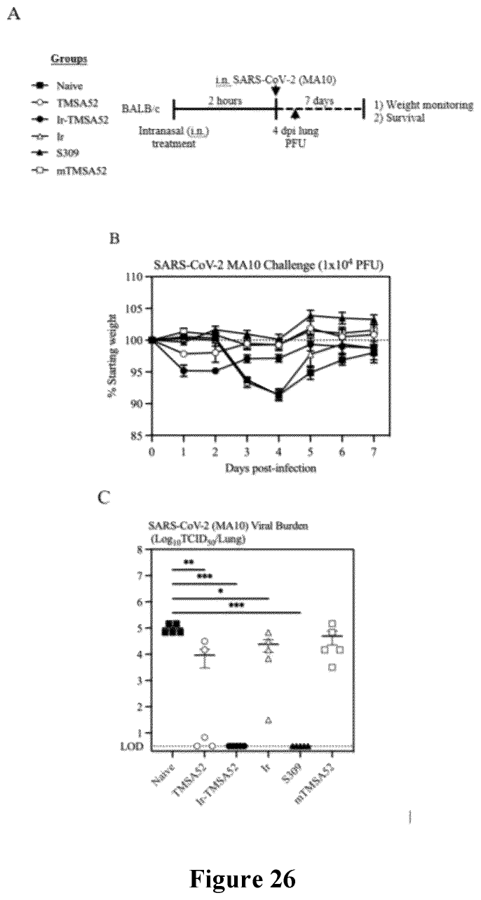

Effective protection against respiratory pathogens such as SARS-COV-2 necessitates the delivery of prophylactic and therapeutic agents directly to the site of infection—the respiratory mucosa. To assess the prophylactic efficacy of our aptamer technology, female BALB/c mice (n=5-10 per group) were treated i.n. with a single bolus containing either trimeric aptamer (TMSA52, open circles), or the iridium nanoplate-scaffolded aptamer (Ir-TMSA52, closed circles) (FIG. 26A). As controls, a subset of animals was either treated with nuclease-free water (closed squares) or received a mutant version of TMSA52 (mTMSA52, open squares) that demonstrates reduced binding affinity to the SARS-COV-2 receptor binding domain (RBD), or the iridium nanoplate alone (Ir, open triangles). As a positive control, a subset of animals was treated with a clinically approved SARS-COV-2 monoclonal antibody (S309, closed triangles). Animals were subsequently i.n. infected with a sublethal dose of a mouse-adapted SARS-COV-2 (1×10⁴ PFU) two hours after treatment. Animals were monitored daily for weight loss for 7 days, with a cohort sacrificed at 4 days post-infection (DPI) to enumerate lung viral burden.

Animals treated with nuclease-free water or those with the iridium nanoplate alone exhibited rapid and continued weight loss up to 4 days post-infection (FIG. 26B), at which animals began to recover. In stark contrast, TMSA52 and Ir-TMSA52 exhibited transient weight loss 2 days post-infection (˜5%), but quickly rebounded to pre-infection weights. No weight loss was observed in animals that received mutant aptamer or the control S309 monoclonal antibody.

To further evaluate the protection provided by i.n. aptamer delivery, viral burden was quantified 4 DPI by a 50% tissue-culture infectious dose (TCID₅₀) assay. As anticipated, animals treated with either the mutant aptamer or the iridium nanoplate alone had similarly high lung viral titers, comparable to nuclease-free water treated control animals (FIG. 26C). In stark contrast, 3/5 animals treated with TMSA52 had a >4 log₁₀ reduction in lung viral burden. Of note, animals that received Ir-TMSA52 had no quantifiable lung viral titers, comparable to animals treated with S309.

### Conclusions

Intranasal delivery of both the trimeric aptamer (TMSA52) and the iridium nanoplate-scaffolded aptamer (Ir-TMSA52) offers robust protection against SARS-COV-2. These treatments effectively mitigate morbidity and substantially reduce lung viral burden. The performance of both TMSA52 and Ir-TMSA52 was comparable to that of the clinically approved SARS-COV-2 monoclonal antibody S309. These findings underscore the potential of our aptamer technology as an effective prophylactic strategy against SARS-COV-2, exhibiting promising congruency to existing clinical standards.

### Example 14: In Vivo Assessment of Aptamers in Protection Against Lethal Challenge with SARS-CoV-2

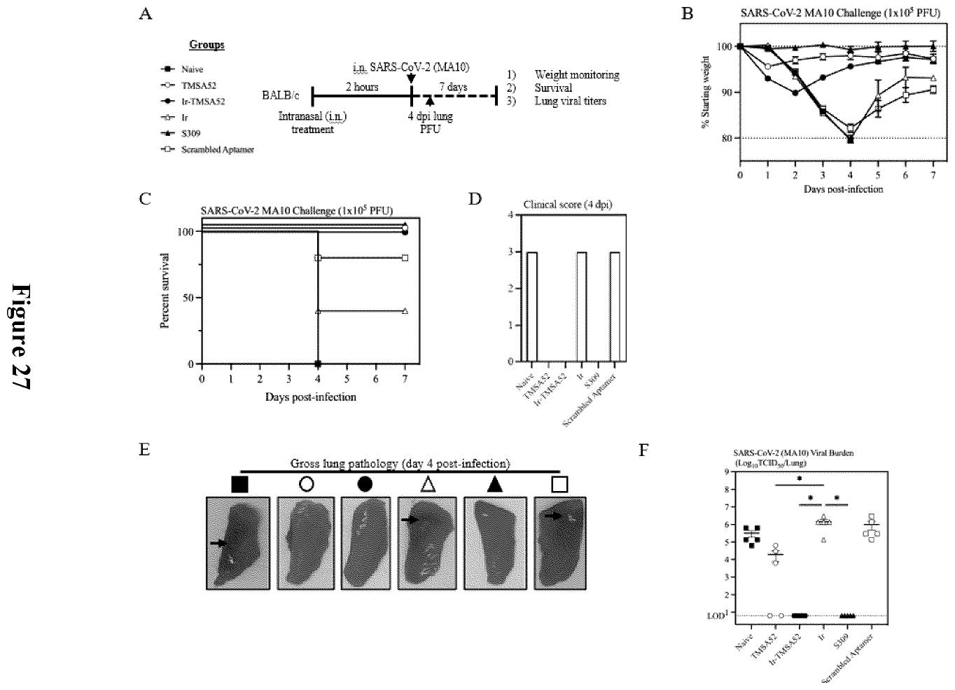

To further assess the prophylactic efficacy of our aptamer technology, female BALB/c mice (n=5-10 per group) were treated i.n. with a single bolus containing either trimeric aptamer (TMSA52, open circles), or the iridium nanoplate-scaffolded aptamer (Ir-TMSA52, closed circles) (FIG. 27A). As controls, a subset of animals was treated with either nuclease-free water, (closed squares), a scrambled aptamer that demonstrates no binding affinity to the SARS-COV-2 RBD (scrambled aptamer, open squares), or the iridium nanoplate alone (Ir, open triangles). As a positive control, a subset of animals was treated with a clinically approved SARS-COV-2 monoclonal antibody (S309, closed triangles). Animals were subsequently i.n. infected with a lethal dose of a mouse-adapted SARS-COV-2 (1×105 PFU), two hours after treatment. Animals were monitored daily for weight loss and clinical signs of disease for 7 days, with a cohort sacrificed at 4 DPI to assess lung pathology and enumerate lung viral burden.

Animals treated with nuclease-free water, the iridium nanoplate, or scrambled aptamer exhibited rapid and continued weight loss, reaching humane endpoint (80% of starting body weight) by 4 DPI (FIG. 27B and C). In agreement with these observations, all animals presented with significant signs of clinical disease, as marked by rapid respiration, hunched posture, and ruffled body condition (FIG. 27D). In stark contrast, TMSA52 and Ir-TMSA52 exhibited transient weight loss 2 DPI (≈10%), but quickly rebounded to pre-infection weights. No weight loss was observed in animals that received the control S309 monoclonal antibody. Neither TMSA52, Ir-TMSA52, nor S309-treated animals presented with any signs of clinical disease by 4 DPI.

To further evaluate the protection provided by i.n. aptamer delivery, lungs were harvested 4 days post-infection for both enumeration of viral burden and gross assessment of lung pathology. In accordance with both weight loss and signs of clinical disease, lungs from animals treated with either nuclease-free water, the iridium nanoplate, or scrambled aptamer exhibited areas of diffuse hemorrhage (Figure. 27E, black arrows). In stark contrast, lungs from animals treated with TMSA52 or Ir-TMSA52 showed no signs of lung pathology and were equivalent to those treated with S309.

Viral burden was subsequently quantified by a TCID50 assay. As expected, animals treated with either the mutant aptamer or the iridium nanoplate alone had similarly high lung viral titers, comparable to nuclease-free water treated control animals (FIG. 27F). In stark contrast, 2/5 animals treated with TMSA52 had a >4 log 10 reduction in lung viral burden. Of note, animals which received Ir-TMSA52 had no quantifiable lung viral titers, comparable to animals treated with S309.

### Conclusions

Intranasal delivery of both the trimeric aptamer (TMSA52) and the aptamer scaffolded on the iridium nanoplate (Ir-TMSA52) offers robust protection against SARS-COV-2. These treatments effectively mitigate morbidity, mortality, lung pathology, and substantially reduce lung viral burden. The performance of both TMSA52 and Ir-TMSA52 was comparable to that of the clinically approved SARS-COV-2 monoclonal antibody S309. These findings underscore the potential of our aptamer technology as an effective prophylactic strategy against SARS-COV-2, exhibiting promising comparability to existing clinical standards.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. A multivalent trident aptamer comprising the general formula: 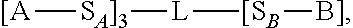
wherein:
A is an aptamer specific to an epitope for binding to a target molecule or protein, wherein said aptamer is 25 to 150 single-stranded nucleotides in length;
L is a trebler biomolecular linker comprising three branches and a root, wherein each branch comprises a terminal end that links to one of the [A-S_{A}]s to form a trident configuration comprising three aptameric branches that cooperatively cross-bind the target molecule or protein so that the target is bound by at least one aptameric branch at any given time;
S_{A} is a spacer molecule that separates each A from the L-branch at a distance of up to 20.5 nm;
B is a functional molecule selected from an aptamer, a reporter molecule, or a crosslinker that is linked to the terminal end of the root of L; and
S_{B} is a spacer molecule that separates B from the L-root at a distance of up to 20.5 nm;
wherein the cooperatively cross-binding trident aptamer synergistically enhances avidity for the target molecule or protein over a corresponding mono-aptomeric form.

2. (canceled)

3. The multivalent trident aptamer of claim 1, wherein the trebler biomolecule is a symmetric trebler biomolecule or an asymmetric trebler molecule.

4. The multivalent trident aptamer of claim 3, wherein the symmetric trebler biomolecule is a trebler phosphoramidite.

5. (canceled)

6. The multivalent trident aptamer of claim 1, wherein S_{A} comprises:
(i) a single stranded nucleic acid sequence of 0 to 30 nucleotides;
(ii) up to 15 linear alkane chains each comprising up to 12 carbon atoms; or
(iii) a linear polyethylene glycol chain of up to 55 ethylene glycol units.

7. The multivalent trident aptamer of claim 6, wherein the (i) single stranded nucleic acid sequence is a single stranded thymine-rich sequence of 12 nucleotides.

8. (canceled)

9. (canceled)

10. The multivalent trident aptamer of claim 1, wherein B is:
(i) an aptamer identical to A, specific to the epitope for binding to the target molecule or protein;
(ii) a reporter molecule selected from an antigen, an enzyme, and a fluorescent molecule; or
(iii) a crosslinker selected from thiol, amide, biotin, digoxigenein, azide, alkyne, carboxyl, and a Click-Chemistry-based crosslinker.

11. (canceled)

12. (canceled)

13. The multivalent trident aptamer of claim 1, wherein S_{B} comprises:
(i) a single stranded nucleic acid sequence of 0 to 30 nucleotides;
(ii) up to 15 linear alkane chains each comprising up to 12 carbon atoms; or
(iii) a linear polyethylene glycol chain of up to 55 ethylene glycol units.

14. The multivalent trident aptamer of claim 13, wherein the (i) single stranded nucleic acid sequence is a single stranded thymine sequence of 5 nucleotides.

15. (canceled)

16. (canceled)

17. The multivalent trident aptamer of claim 1, wherein the trident aptamer is a homo-trimeric aptamer, wherein the As are identical and are each specific to the same epitope for binding to the target molecule or protein.

18. The multivalent trident aptamer of claim 17, wherein the As are in the same 5′ to 3′ orientation relative to L or wherein the As are in the same 3′ to 5′ orientation relative to L.

19. (canceled)

20. The multivalent trident aptamer of claim 1, wherein the trident aptamer is a hetero-trimeric aptamer, wherein the As are each specific to a different epitope for binding to the target molecule or protein.

21. The multivalent trident aptamer of claim 1, wherein A is between 25 to 90 nucleotides in length.

22. The multivalent trident aptamer of claim 1, wherein the target molecule is:
(i) SARS-COV-2 S protein;
(ii) Influenza HA;
(iii) VEGF165; or
(iv) Troponin I.

23. The multivalent trident aptamer of claim 22, wherein the target molecule is SARS-COV-2 S protein and A is MSA52T8.

24. (canceled)

25. The multivalent trident aptamer of claim 22, wherein the target molecule is Influenza HA and A is RHA06.

26. (canceled)

27. The multivalent trident aptamer of claim 22, wherein the target molecule is VEGF165 and A is H1A.

28. (canceled)

29. The multivalent trident aptamer of claim 22, wherein the target molecule is Troponin I and A is TnAp1.

30. A pharmaceutical composition comprising the multivalent trident aptamer of claim 1 and one or more pharmaceutically acceptable excipients.

31. (canceled)

32. A biosensor comprising the multivalent trident aptamer of claim 1 immobilized on and/or in a material.

33. A method for detecting the presence of a target molecule in a sample, the method comprising:
a. Contacting the sample with the multivalent trident aptamer of claim 1, wherein the multivalent trident aptamer binds the target molecule; and
b. Detecting the binding of the multivalent trident aptamer with the target molecule.


SEQUENCE LISTING (25 sequences)

SEQ ID NO: 1 - DNA, 79 bp, synthetic construct
  source 1..79: mol_type = other DNA
ttacgtcaaggtgtcactccgtagggtttggctccgggcctggcgtcggtcgtctctcgcgaagcatctctttggcgtg

SEQ ID NO: 2 - DNA, 84 bp, synthetic construct
  source 1..84: mol_type = other DNA
tttttttacgtcaaggtgtcactccgtagggtttggctccgggcctggcgtcggtcgtctctcgcgaagcatctctttggcgtg

SEQ ID NO: 3 - DNA, 178 bp, synthetic construct
  source 1..178: mol_type = other DNA
ttacgtcaaggtgtcactccgtagggtttggctccgggcctggcgtcggtcgtctctcgcgaagcatctctttggcgtgttttttttttttttttttttttacgtcaaggtgtcactccgtagggtttggctccgggcctggcgtcggtcgtctctcgcgaagcatctctttggcgtg

SEQ ID NO: 4 - DNA, 183 bp, synthetic construct
  source 1..183: mol_type = other DNA
tttttttacgtcaaggtgtcactccgtagggtttggctccgggcctggcgtcggtcgtctctcgcgaagcatctctttggcgtgttttttttttttttttttttttacgtcaaggtgtcactccgtagggtttggctccgggcctggcgtcggtcgtctctcgcgaagcatctctttggcgtg

SEQ ID NO: 5 - DNA, 94 bp, synthetic construct
  source 1..94: mol_type = other DNA
ttacgtcaaggtgtcactccgtagggtttggctccgggcctggcgtcggtcgtctctcgcgaagcatctctttggcgtgttttttttttttttt

SEQ ID NO: 6 - DNA, 94 bp, synthetic construct; identical to SEQ ID NO: 5
  source 1..94: mol_type = other DNA

SEQ ID NO: 7 - DNA, 40 bp, synthetic construct
  source 1..40: mol_type = other DNA
gcgagagacgaccgacgccaggcccggagccaaaccctac

SEQ ID NO: 8 - DNA, 49 bp, synthetic construct
  source 1..49: mol_type = other DNA
cgaagcatctcgtgtcactccgtagggtttggctccgggcctggcgtcg

SEQ ID NO: 9 - DNA, 61 bp, synthetic construct
  source 1..61: mol_type = other DNA
cgaagcatctcgtgtcactccgtagggtttggctccgggcctggcgtcgtttttctctcgc

SEQ ID NO: 10 - DNA, 61 bp, synthetic construct
  source 1..61: mol_type = other DNA
cgaagcatctcgtgtcactcgagctggtttggcggcctgcctggcgtcgtttttctctcgc

SEQ ID NO: 11 - DNA, 48 bp, synthetic construct
  source 1..48: mol_type = other DNA
gggtttgggttgggttgggtttttgggtttgggttgggttgggaaaaa

SEQ ID NO: 12 - DNA, 60 bp, synthetic construct
  source 1..60: mol_type = other DNA
gggtttgggttgggttgggtttttgggtttgggttgggttgggaaaaatttttctctcgc

SEQ ID NO: 13 - DNA, 60 bp, synthetic construct
  source 1..60: mol_type = other DNA
gtttggttatgggagtgtggggggaggttggtggtgtgtggttattattttttctctcgc

SEQ ID NO: 14 - DNA, 28 bp, synthetic construct
  source 1..28: mol_type = other DNA
gcccgtcttccagacaagagtgcagggc

SEQ ID NO: 15 - DNA, 40 bp, synthetic construct
  source 1..40: mol_type = other DNA
gcccgtcttccagacaagagtgcagggctttttctctcgc

SEQ ID NO: 16 - DNA, 80 bp, synthetic construct
  source 1..80: mol_type = other DNA
ggcagcaggaagacaagacatgggtggcggggacggggcgatgggaacttagattgctagtggttctgtggttgctctgt

SEQ ID NO: 17 - DNA, 92 bp, synthetic construct
  source 1..92: mol_type = other DNA
ggcagcaggaagacaagacatgggtggcggggacggggcgatgggaacttagattgctagtggttctgtggttgctctgttttttctctcgc

SEQ ID NO: 18 - DNA, 49 bp, synthetic construct
  source 1..49: mol_type = other DNA
ctttccggttaatttatgctctacccgtccacctaccggaaagcatctc

SEQ ID NO: 19 - DNA, 49 bp, synthetic construct
  source 1..49: mol_type = other DNA
cttccacgggtttggcgtcgggcctggcggggggatagtgcggtggaag

SEQ ID NO: 20 - DNA, 49 bp, synthetic construct; identical to SEQ ID NO: 8
  source 1..49: mol_type = other DNA

SEQ ID NO: 21 - DNA, 12 bp, synthetic construct
  source 1..12: mol_type = other DNA
tttttctctcgc

SEQ ID NO: 22 - DNA, 183 bp, synthetic construct
  source 1..183: mol_type = other DNA
ctttccggttaatttatgctctacccgtccacctaccggaaagcatctctttttctctcgccttccacgggtttggcgtcgggcctggcggggggatagtgcggtggaagtttttctctcgccgaagcatctcgtgtcactccgtagggtttggctccgggcctggcgtcgtttttctctcgc

SEQ ID NO: 23 - DNA, 22 bp, synthetic construct
  source 1..22: mol_type = other DNA
gcgagagaaaaagagatgcttt

SEQ ID NO: 24 - DNA, 22 bp, synthetic construct
  source 1..22: mol_type = other DNA
gcgagagaaaaacttccaccgc

SEQ ID NO: 25 - DNA, 22 bp, synthetic construct
  source 1..22: mol_type = other DNA
gcgagagaaaaacgacgccagg